(19) United States
(12) Patent Application Publication
(10) Pub. No.: US 2004/0204477 A1
(43) Pub. Date: Oct. 14, 2004

(54) Interaction inhibitors of tcf-4 with beta-catenin

(76) Inventors: Juergen Moll, Como (IT); Stefan Knapp, Monza (IT); Claudio Dalvit, Milan (IT); Jean-Yves Trosset, Varese (IT); Michael Sundstrom, Stockholm (SE); Sergio Mantegani, Milan (IT)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/482,755

(22) PCT Filed: Jul. 3, 2002
(86) PCT No.: PCT/EP02/07536

(30) Foreign Application Priority Data
Jul. 9, 2001 (EP) 01202626.6

(51) Int. Cl. A61K 31/40; A61K 31/18; A61K 31/16

(52) U.S. Cl. 514/426; 514/602; 514/613

(57) ABSTRACT

A compound of formula (I) is provided which is able to interact with β-catenin/TCF-4 binding site, having a structure essentially equivalent to a pharmacophore (IA), as herein described.

### FIELD OF THE INVENTION

The invention provides a compound of formula (I) as herein defined, which is able to interact with β-catenin/TCF-4 binding site, having a structure essentially equivalent to a pharmacophore (IA), as herein defined.

The compounds of formula (I) are useful as modulating agents for inhibiting β-catenin mediated gene expression. Accordingly, they can be used as therapeutic agents, e.g. as antiproliferative agents, in particular, in preventing and treating cancer, in inhibiting cancer metastasis in a patient, in treating Alzheimer's disease and in modulating hair growth.

### DESCRIPTION OF THE RELATED ART

The Wnt signal pathway plays a role in diverse cellular processes such as migration, differentiation and proliferation (For Review see e.g. Bienz M. & Clevers H., Linking colorectal cancer to Wnt signalling. Cell 103:311-20, 2000). According to the current view in the absence of Wnt signalling, a complex consisting of Axin, APC, the serine/threonine kinase GSK3β and β-catenin is formed. As a consequence β-catenin is phosphorylated by GSK3β which leads to ubiquitination by the SCF complex containing the F-box protein βTrCP/Slimb. As a result β-catenin is degraded by the proteasome (Jiang J. & Struhl G. 1998, Regulation of the Hedgehog and Wingless signalling pathways by the F-box/WD40-repeat protein Slimb. Nature, 391:493-6, 1998; Marikawa Y. & Elinson RP, beta-TrCP is a negative regulator of Wnt beta-catenin signalling pathway and dorsal axis formation in Xenopus embryos. Mech Dev 77:75-80, 1998). Degradation of β-catenin is further enhanced by GSK3β mediated phosphorylation of APC which causes loss of affinity for β-catenin.

Upon stimulation by Wnt ligands to its receptors (Frizzled), the cytoplasmic protein Dishevelled is recruited to the membrane and activates Frat-1, which negatively regulates GSK3β. As a consequence β-catenin lacks phosphorylation at critical residues and escapes degradation. β-catenin is translocated to the nucleus where it interacts with transcription factors of the LEF-1/TCF family and regulates expression of specific genes towards LEF-1/TCF transcription factors are able to bind DNA consensus sequences via their HMG-domain. However, they need Co-activators such as β-catenin to activate gene transcription. The corresponding target genes are known to be involved in several aspects of human cancer and include c-myc (He T. C. et al., Identification of c-MYC as a target of the APC pathway. Science 281:1509-12,1998), cyclin D1 (Shtutman M. et al., The cyclin D1 gene is a target of the beta-catenin/LEF-1 pathway. Proc NatI Acad Sci USA. 96(10):5522-7, 1999), gastrin (Koh T. J. et al., Gastrin is a target of the beta-catenin/TCF-4 growth-signalling pathway in a model of intestinal polyposis. J Clin Invest. 106:533-9, 2000) the matrix metalloproteinase MMP-7 (Brabletz T. et al., beta-catenin regulates the expression of the matrix metalloproteinase-7 in human colorectal cancer. Am J Pathol 155:1033-8, 1999) and MDR-1 (Yamada T. et al., Transactivation of the multidrug resistance 1 gene by T-cell factor 4/beta-catenin complex in early colorectal carcinogenesis. Cancer Res 60:4761-,2000). All these target genes have been shown to be regulated by TCF-4 a specific member of the LEF1/TCF family and might play a role during cancer development and progression. Hence, the interaction of TCF-4 with β-catenin is seen one of the crucial events in particular during colorectal tumorigenesis. Over-expression of dominant negative TCF-4 in colorectal tumor cells causes cells to arrest in the G1 phase of the cell cycle supporting the relevance of TCF-4 in tumor cell proliferation (Tetsu O. & McCormick F., Beta-catenin regulates expression of cyclin D1 in colon carcinoma cells. Nature, 398: 422-6, 1999). Hence, the interaction of β-catenin and TCF-4 represents a promising target for therapeutic intervention in cancer and small molecular weight inhibitors of this interaction might have anti-tumorigenic effects. Some 85% of all sporadic and hereditary colorectal tumors show loss of APC function, which results in stabilization of β-catenin (Kinzler K. W. & Vogelstein B., Lessons from hereditary colorectal cancer. Cell 87: 159-70, 1996). Among the colorectal tumors not bearing a mutation in APC, most carry a mutation in β-catenin. These mutations are located preferentially within the four serine/threonine phosphorylation sites which are the target of GSK3β. Mutations in the Wnt pathway were found in other tumors including hepatocellular carcinomas, melanomas, gastric cancer or hair follicle tumors (Reviewed in Polakis P., Wnt signalling and cancer, Genes & Dev 14:1837-1851, 2000). All these alterations finally render β-catenin refractory to the ubiquitin-mediated destruction and result in nuclear translocation. Subcellular localization of β-catenin is critically regulated by APC, probably based on sequestration of β-catenin from the LEF-1/TCF transcription complex (Neufeld K. L., et al., EMBO Reports, 1, 519-523, 2000). Mutant APCs, which lack nuclear localization signals (NLS) or nuclear export signals (NES) are not able to keep low nuclear β-catenin levels (Henderson B. R., Nuclear-cytoplasmic shuttling of APC regulates, β-catenin subcellular localization and turnover, Nature Cell Biology, 2, 653-660, 2000; Rosin-Arbesfeld R. et al., The APC tumour suppressor has a nuclear export function. Nature, 406:1009-12, 2000). A core region of β-catenin, composed of 12 copies of a 42 amino acid sequence motif known as armadillo repeat, mediates the protein-protein interactions with LEF-1/TCF family transcription factors. The three-dimensional structure of the armadillo repeat region has been determined (Huber A. H. et al., Three-dimensional structure of the armadillo repeat region of β-catenin. Cell 90:871-82, 1997) and revealed that the repeats form a superhelix of helices that features a long, positively charged groove. Amino acid residues in β-catenin which are crucial for binding to LEF-1 and TCF have been identified and define a hot spot along the armadillo superhelix. The essential amino acid residues of β-catenin for interaction with LEF-1 flank a hydrophobic pocket in the region around Leu427 (von Kries J. P. et al., Hot spots in beta-catenin for interactions with LEF-1, conductin and APC. Nat Struct Bio 19:800-7, 2000).

### DESCRIPTION OF THE INVENTION

The invention provides a compound of formula (I) which is able to interact with β-catenin/TCF-4 binding site, having a structure essentially equivalent to a pharmacophore (IA), characterized by a structure which comprises:

a saturated, partially saturated, carbocyclic or heteroaromatic pentatomic ring (A), substituted at least by a substituent (Z) and optionally by a substituent R as herein defined; or substituents (Z) and R, taken together, form an optionally substituted, partially saturated monocyclic or bicyclic ring system;

an optionally substituted, saturated, partially saturated, carbocyclic, aromatic or internally condensed ring (B); rings (A) and (B) being separated by a spacer (Y) which provides an inter-center distance between rings (A) and (B) of about 10.9±2 Angstrom; wherein the relative orientation between said rings (A) and (B) is such that the angle θ between the two centroid vectors is about 40 degrees ±30 degrees; the convention for the orientation of the two vectors being such that cos θ is >0.

According to a preferred embodiment of the invention, when substituent (Z) is a small group like hydrogen, an halogen atom, methyl, methoxy, hydroxy, cyano or amino the distance between substituent (Z) and the center of ring (A) is about from 2.3 Angstrom to 2.9 Angstrom, and the distance between substituent (Z) and the center of ring (B) is about from 13 Angstrom to 13.5 Angstrom.

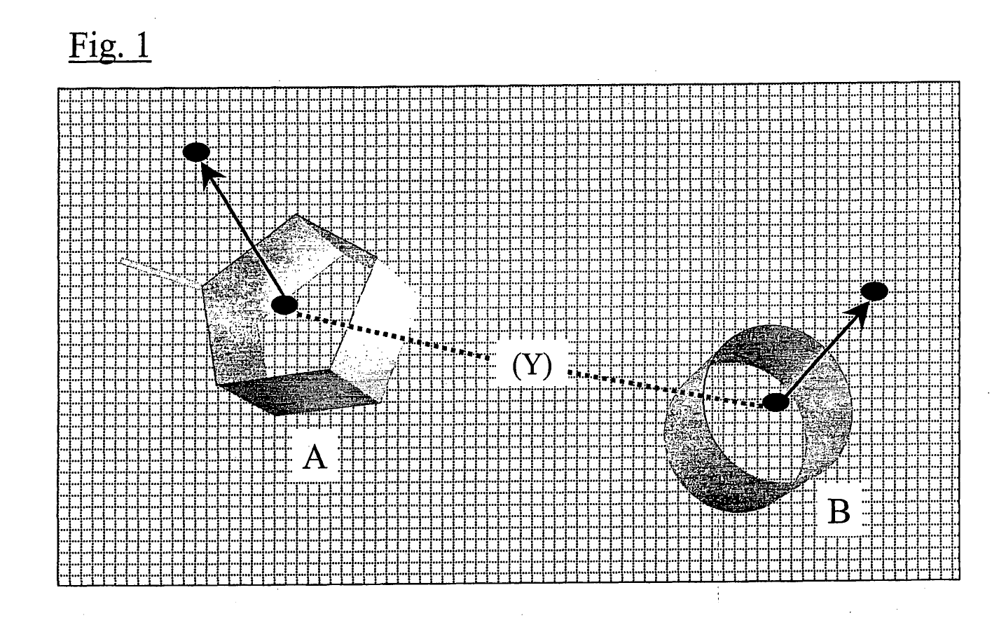

FIG. 1 is a graphic representation of the pharmacophore (IA), which is the first object of the invention and is characterized by the above features.

The invention also provides a screening method for identifying a candidate drug for use in Familial Adenomatous Polyposis (FAP) patients, patients with APC or β-catenin mutations, or patients with increased risk of developing cancer, comprising the steps of determining the optimal fit of a plurality of compounds into pharmacophore (IA), as defined above, such that the lowest energy of interaction and the best steric fit are obtained.

Accordingly, the invention also provides the use of a compound as identified by the above screening method in the preparation of a medicament which is able to interact with β-catenin/TCF-4 binding site.

In a further aspect, the invention provides a β-catenin/TCF-4 interaction modulating, in particular an interaction inhibitor, compound capable of adopting a structure having a pharmacophoric pattern essentially equivalent to the pharmacophoric pattern of pharmacophore (IA), as defined above.

Accordingly, the invention provides a compound (I) or a pharmaceutically acceptable salt thereof, which is able to interact with β-catenin/TCF-4 binding site having the following formula

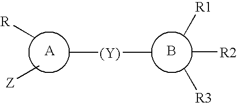

wherein:

(A) is a saturated, partially saturated, carbocyclic or heteroaromatic pentatomic ring;

(B) is a saturated, partially saturated, carbocyclic, aromatic or internally condensed ring;

(Y), in its shortest way, is a spacer consisting of about 4 to 9 chain atoms chosen independently from C, O, N and S, which may have independently different hybridization states (e.g. sp3, sp2 or sp), and wherein two to five adjacent atoms of the chain my be part of an optionally substituted aryl, heteroaryl or partially saturated aryl or heteroaryl ring system, which may be either isolated or include ring (3).

Z is a substituent selected independently from hydrogen, halogen, hydroxy, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl;

R is independently selected from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl; or Z and R, taken together, form an optionally substituted, partially saturated monocyclic or bicyclic ring system;

each of R1, R2 and R3, which may be independently the same or different, is chosen from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl; a NHCORc or NHSO2Rc group wherein Rc is C1-C4 alkyl; and a C5-C6 cycloalkyl-oxy or aryloxy group.

All the possible stereoisomers, and mixtures thereof, of the compounds of formula (I) are also object of the invention.

A saturated ring (A) may be for instance a cyclopentyl ring or a saturated heterocyclic ring containing from 1 to 3 heteroatoms chosen from N, O and S, for instance pyrrolidine.

An heteroaromatic pentatomic ring (A) may be for instance an heterocyclic ring containing from 1 to 3 heteroatoms chosen from N, O and S; for instance furane, thiazole, thiadiazole, thiophene, isoxazole, triazole, pyrrole, imidazole, oxazole and oxadiazole.

When Z and R, taken together, form an optionally substituted, partially saturated monocyclic or bicyclic ring system, such a ring system can be for instance a partially saturated phenyl or naphthyl ring, optionally substituted by one or two substituents chosen independently from halogen, hydroxy, amino, C1-C4 alkyl and C1-C4 alkoxy. Ring (A) and the condensed partially saturated naphthyl ring can thus provide for instance an optionally substituted 4,5-dihydronaphtho[1,2-d][1,3]thiazol-2-yl or 4,5-dihydro-3H-naphtho[1,2-d]imidazol-2-yl ring system.

A saturated ring (B) may be for instance a C3-C7 cycloalkyl ring or a C5-C7 saturated heterocyclic ring containing from 1 to 3 heteroatoms chosen from N, O and S. Preferred examples of C3-C7 cycloalkyl rings are cyclopentyl, cyclohexyl and cycloheptyl. Preferred examples of C5-C7 saturated heterocyclic rings are pyrrolidine, piperazine, piperidine, morpholino and hexahydroazepine.

An aromatic ring (B) may be a C6-C13 aryl or C5-C6 heteroaryl ring containing from 1 to 3 heteroatoms chosen from N, O and S. Preferred examples of aryl rings are phenyl and naphthyl. Preferred examples of heteroaryl rings are furane, thiazole, thiadiazole, thiophene, isoxazole, triazole, oxadiazole, pyridine, pyrrole, thiophene, oxazole, isoxazole, imidazole, pyrimidine, pyridazine, pyrazine, quinoline, isoquinoline, benzothiazole, benzoimidazole and benzoxazole. More preferably, furane, thiazole, thiadiazole, thiophene, isoxazole, triazole, oxadiazole and pyridine.

A partially saturated ring (B) may be for instance a partially saturated C4-C9 atom ring system in which 1 to 3 carbon atoms are optionally replaced by an heteroatom chosen from O, S and N. Preferred examples are cyclohexene, piperideino, tetrahydroquinoline, tetrahydroisoquinoline and dihydropyrrole.

An internally condensed ring (B) may a group of formula (C)

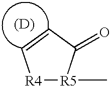

wherein (D) may complete a phenyl ring or be absent; each of R4 and R5 may be a OH or N(HRd) group, wherein Rd is C1-C4 alkyl, thus providing an internal hydrogen bridge between R4 and R5. Preferred examples of such internally condensed rings (B) are those provided by ortho-substituted salicylic or anthranylic acid derivatives.

A spacer (Y) is for example selected from:

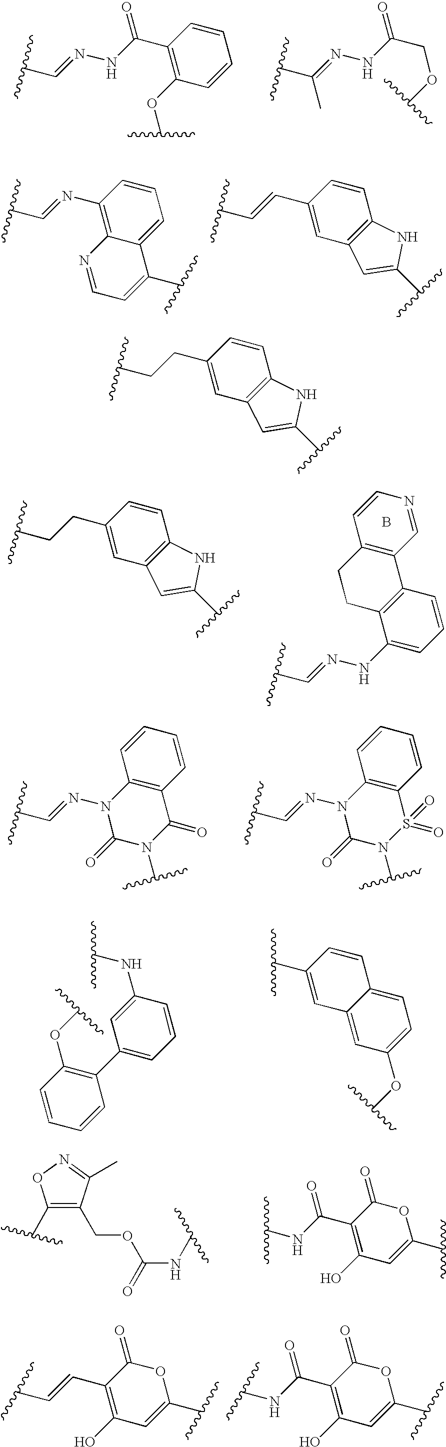

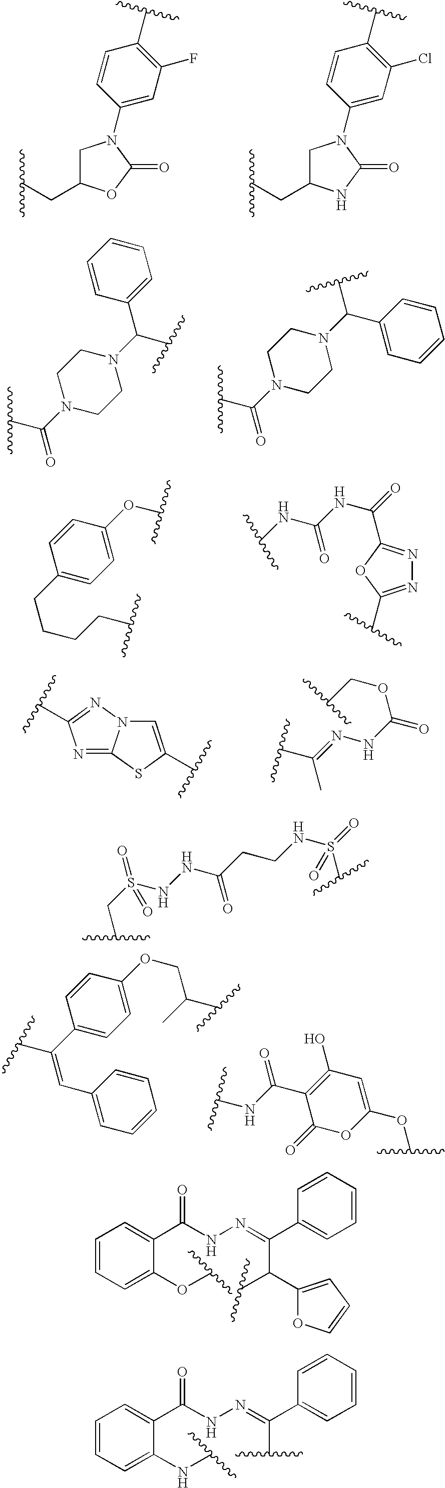

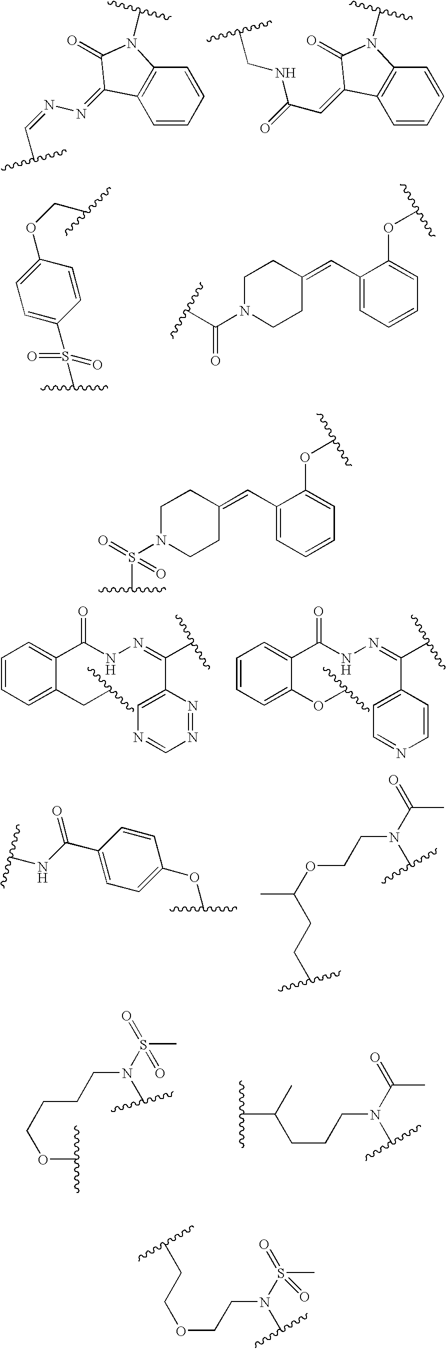

It will be appreciated that the above specific examples of spacer (Y) also illustrate an example of spacer (Y), in which two to five adjacent atoms of the chain are part of an optionally substituted aryl, heteroaryl or partially saturated aryl or heteroaryl ring system, which may be either isolated or include ring (B).

When spacer (Y) contains an optionally substituted aryl, heteroaryl or partially saturated aryl or heteroaryl ring system, such a ring system may be substituted by one to three substituents selected independently from halogen, cyano, oxo, hydroxy, carboxy, carboxy(C1-C4 alkyl), a straight or branched C1-C4 alkyl group optionally substituted by 1 to 3 halogen atoms or by phenyl, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl. A halogen atom is e.g. fluorine, chlorine or bromine, in particular fluorine and chlorine. A C1-C4 alkyl group is e.g. methyl, ethyl, propyl, isopropyl, butyl and isobutyl, in particular methyl, ethyl and propyl.

A C1-C4 alkyl group substituted by 1 to 3 halogen atoms is e.g. trifluoromethyl.

A C1-C4 alkoxy group is e.g. methoxy, ethoxy, propoxy, isopropoxy or butoxy, preferably methoxy or ethoxy.

A pharmaceutically acceptable salt of a compound of formula (I) may be for example the acid addition salts with inorganic or organic, e.g. nitric, hydrochloric, hydrobromic, sulphuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

Accordingly, the invention provides the use of a compound of formula (I) or a pharmaceutically acceptable salt thereof, as herein defined, in the preparation of a pharmaceutical composition, which interacts with the β-catenin/TCF-4 interaction.

According to a preferred aspect of the invention “interaction” results in modulation, in particular inhibition, of β-catenin/TCF-4 binding. Therefore the compound of the invention are particularly useful in preventing and treating proliferative disorders, including cancer, in particular in PAF patients, in patients with APC or β-catenin mutations or patients with increased risk of developing cancer, in inhibiting cancer metastasis, in treating Alzheimer's disease and in modulating hair growth. Examples of such cancers are colorectal carcinoma, melanoma, liver carcinoma, breast cancer and prostate cancer.

The invention therefore provides the use of a compound of formula (I) or a pharmaceutically acceptable salt thereof, having the following formula:

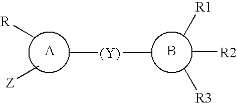

wherein:

(A) is a saturated, partially saturated, carbocyclic or heteroaromatic pentatomic ring;

(B) is a saturated, partially saturated, carbocyclic, aromatic or internally condensed ring;

(Y), in its shortest way, is a spacer consisting of about 4 to 9 chain atoms chosen independently from C, O, N and S, which may have independently different hybridization states (e.g. sp3, sp2 or sp), and wherein two to five adjacent atoms of the chain may be part of an optionally substituted aryl, heteroaryl or partially saturated aryl or heteroaryl ring system, which may be either isolated or include ring (B).

Z is a substituent selected independently from hydrogen, halogen, hydroxy, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl;

R is independently selected from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl; or Z and R, taken together, form an optionally substituted, partially saturated monocyclic or bicyclic ring system; or Z and R, taken together, form an optionally substituted, partially saturated monocyclic or bicyclic ring system;

each of R1, R2 and R3, which may be independently the same or different, is chosen from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl; a NHCORc or NHSO2Rc group wherein Rc is C1-C4 alkyl; and a C5-C6 cycloalkyl-oxy or aryloxy group, in the preparation of a pharmaceutical composition, for use in inhibiting β-catenin/TCF-4 interaction.

In particular, the invention provides the use of a compound of formula (I) or a pharmaceutically acceptable salt thereof, as herein defined, in the preparation of a pharmaceutical composition, for use in preventing and treating proliferative disorders, including cancer, in inhibiting cancer metastasis, in treating Alzheimer's disease and in modulating hair growth.

More specifically, the invention provides the use of a compound of formula (I) or a pharmaceutically acceptable salt thereof, as herein defined, in the preparation of a pharmaceutical composition, for use in preventing and treating colorectal carcinoma, melanoma, liver carcinoma, breast cancer and prostate cancer.

The invention also provides a compound of formula (I) or a pharmaceutically acceptable salt thereof, as herein defined, for use as a medicament, provided that such compound is other than N′-[(E)-(5-methyl-2-furyl)methylidene]-2-phenoxybenzohydrazide.

The invention also provides a compound of formula (I) or a pharmaceutically acceptable salt thereof, as herein defined, for use in modulating, in particular in inhibiting, β-catenin/TCF-4 interaction, provided that such compound is other than N′-[(E)-(5-methyl-2-furyl)methylidene]-2-phenoxybenzohydrazide.

The invention also provides a compound of formula (I) or a pharmaceutically acceptable salt thereof, as herein defined, for use in preventing and treating proliferative disorders, including cancer, in inhibiting cancer metastasis, in treating Alzheimer's disease and in modulating hair growth, with the exception of compound N′-[(E)-(5-methyl-2-furyl)methylidene]-2-phenoxybenzohydrazide.

The invention also provides a compound of formula (I) or a pharmaceutically acceptable salt thereof, as herein defined, for use in preventing and treating colorectal carcinoma, melanoma, liver carcinoma, breast cancer and prostate cancer, with the exception of compound N′-[(E)-(5-methyl-2-furyl)methylidene]-2-phenoxybenzohydrazide.

The invention also provides a method for modulating, in particular inhibiting, β-catenin/TCF-4 interaction in a patient in need thereof, the method comprising administering to said patient a therapeutically effective amount of a compound of formula (I) or a pharmaceutically acceptable salt thereof.

The invention also provides a method for preventing and treating proliferative disorders, including cancer, in inhibiting cancer metastasis, in treating Alzheimer's disease and in modulating hair growth, in a patient in need thereof, the method comprising administering to said patient a therapeutically effective amount of a compound of formula (I) or a pharmaceutically acceptable salt thereof.

The invention also provides a method for preventing and treating colorectal carcinoma, melanoma, liver carcinoma, breast cancer and prostate cancer, in a patient in need thereof, the method comprising administering to said patient a therapeutically effective amount of a compound of formula (I) or a pharmaceutically acceptable salt thereof.

The method according to the invention is particularly useful in Familial Adenomatous Polyposis (FAP) patients, patients with APC or β-catenin mutations, and patients with increased risk of developing cancer.

The invention also provides a novel compound of formula (I) or a pharmaceutically acceptable salt thereof, as herein defined, with the exception of compound N′-[(E)-(5-methyl-2-furyl)methylidene]-2-phenoxybenzohydrazide.

Compound N′-[(E)-(5-methyl-2-furyl)methylidene]-2-phenoxybenzohydrazide is a known compound. It is compound No. 320 (i.e. PNU-74654) of WO 87/06127.

The invention also provides a pharmaceutical composition comprising a compound of formula (I) or a pharmaceutically acceptable salt thereof, as herein defined, with the exception of compound N′-[(E)-(5-methyl-2-furyl)methylidene]-2-phenoxybenzohydrazide, as active ingredient and a pharmaceutically acceptable carrier and/or diluent.

Preferred compounds of formula (I) are those wherein:

(A) is a ring selected from cyclopentyl, pyrrolidine, furane, pyrrole, thiophene, oxazole, isoxazole, imidazole, thiazole, oxadiazole, thiadiazole and triazole.

(B) is a ring selected from cyclopentyl, cyclohexyl, cycloheptyl, pyrrolidine, piperazine, piperidine, morpholino, hexahydroazepine, cyclohexene, piperideino, tetrahydroquinoline, tetrahydroisoquinoline, dihydropyrrole, phenyl, naphthyl, furane, pyrrole, thiophene, oxazole, isoxazole, imidazole, thiazole, oxadiazole, thiadiazole, triazole, pyridine, pyrimidine, pyridazine, pyrazine, quinoline, isoquinoline, benzothiazole, benzoimidazole and benzoxazole;

spacer (Y) is selected from

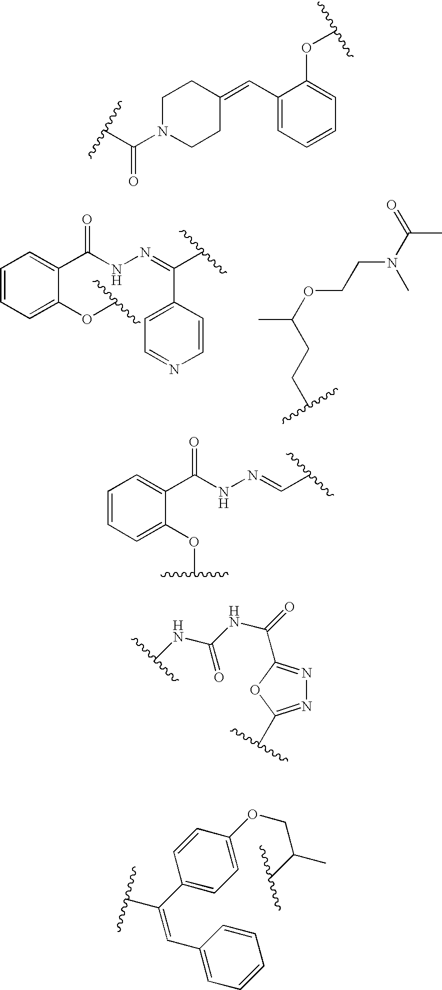

Z is a substituent selected from hydrogen, halogen, hydroxy, cyano, C1-C4 alkyl, trifluoromethyl, C1-C4 alkoxy, amino, methyl-amino, ethylamino, dimethyl-amino, diethylamino, NHCO-ethyl and NHSO2-methyl.

R is from hydrogen, halogen, cyano, C1-C4 alkyl, trifluoromethyl, C1-C4 alkoxy, amino, methylamino, ethylamino, dimethylamino, diethylamino, NHCO-ethyl and NHSO2-methyl; or Z and R, taken together, form a partially saturated phenyl or naphthalene ring;

each of R1, R2 and R3 is independently chosen from hydrogen, halogen, cyano, C1-C4 alkyl, trifluoromethyl, C1-C4 alkoxy, amino, methylamino, ethylamino, dimethyl-amino, diethylamino, NHCO-ethyl, NHSO2-methyl, cyclopentyloxy and cyclohexyloxy.

More preferred compounds of formula (I) are those wherein:

(A) is a ring selected from furane, thiadiazole, isoxazole, thiophene, pyrrolidine, triazole, oxadiazole and thiazole;

(B) is a ring selected from furane, pyridine, phenyl, morpholine, isoxazole, pyrrolidine and thiazole;

spacer (Y) is selected from

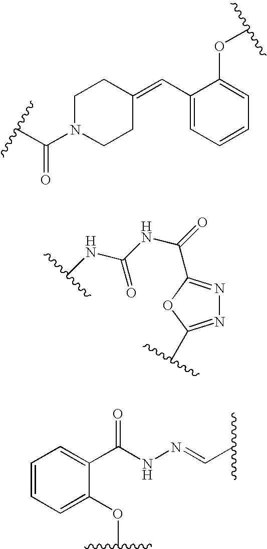

substituent (Z) is halogen, amino, hydroxy, C1-C4 alkyl and C1-C4 alkoxy;

R is hydrogen; or Z and R, taken together with ring (A) form a 4,5-dihydronaphtho[1,2-d][1,3]thiazol-2-yl or 4,5-dihydro-3H-naphtho[1,2-d]imidazol-2-yl ring system;

each of R1, R2 and R3 is independently chosen from hydrogen, amino, hydroxy, C1-C4 alkyl and C1-C4 alkoxy.

Specific examples of compounds of formula (I) are the following:

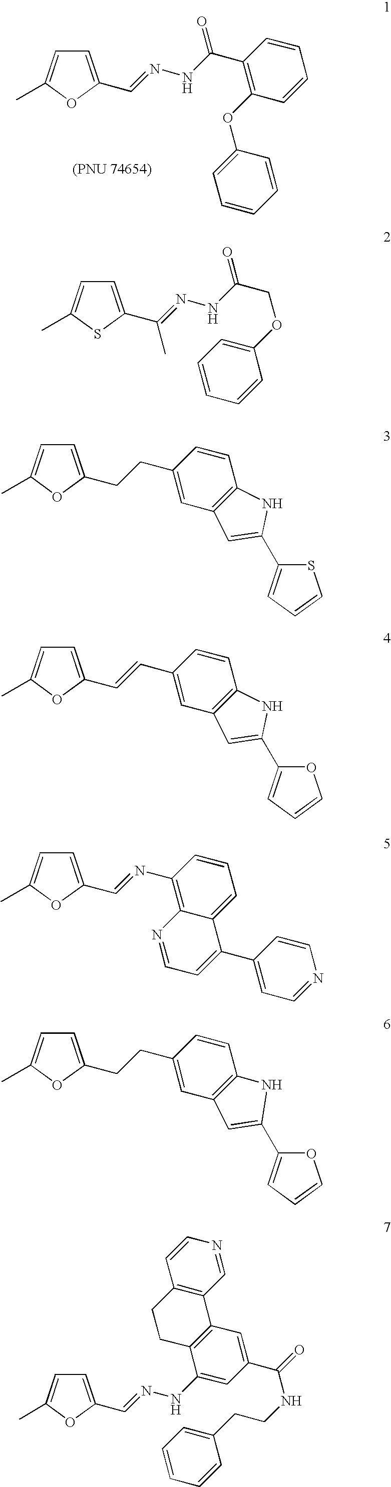

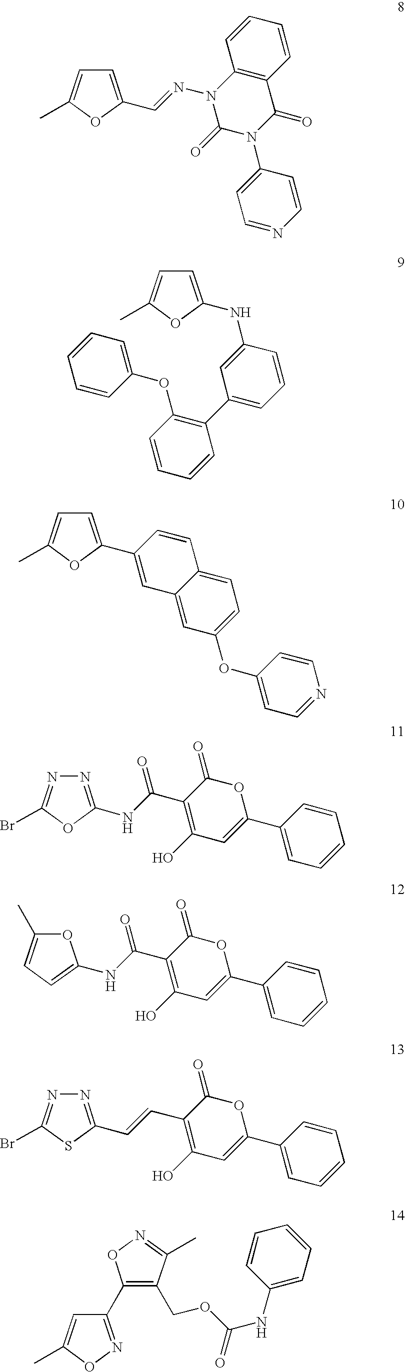

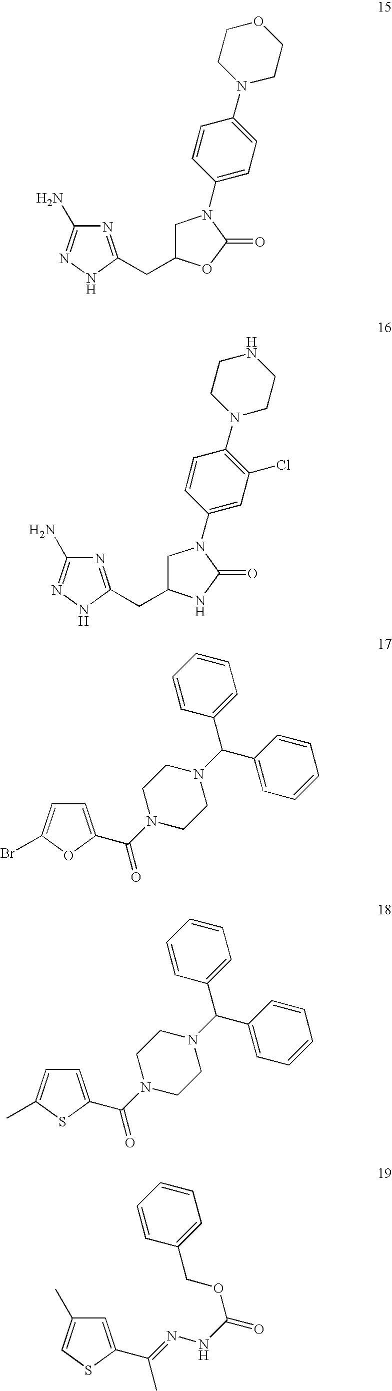

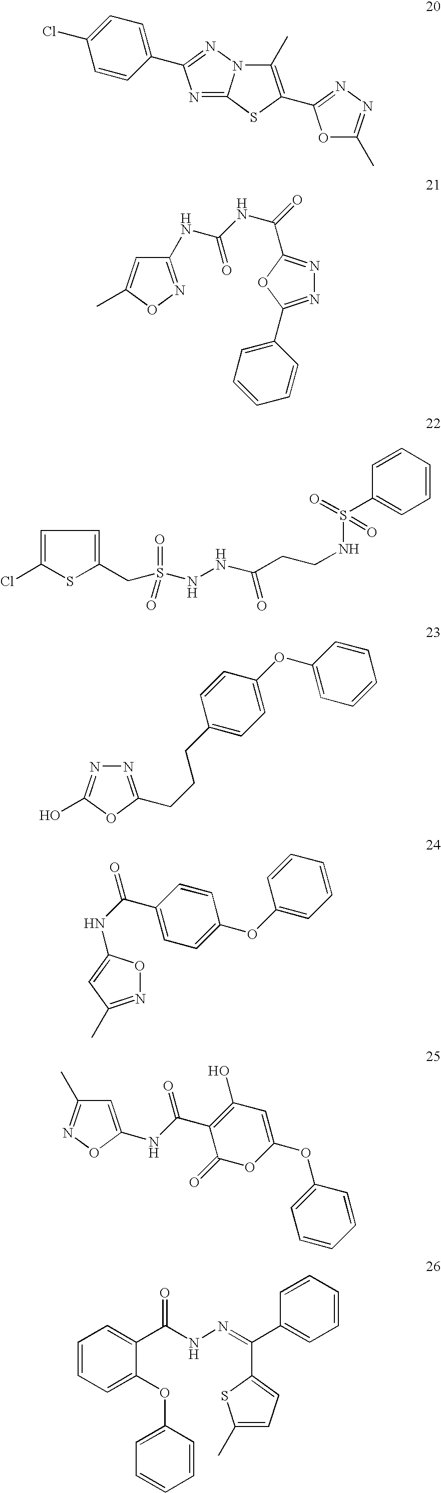

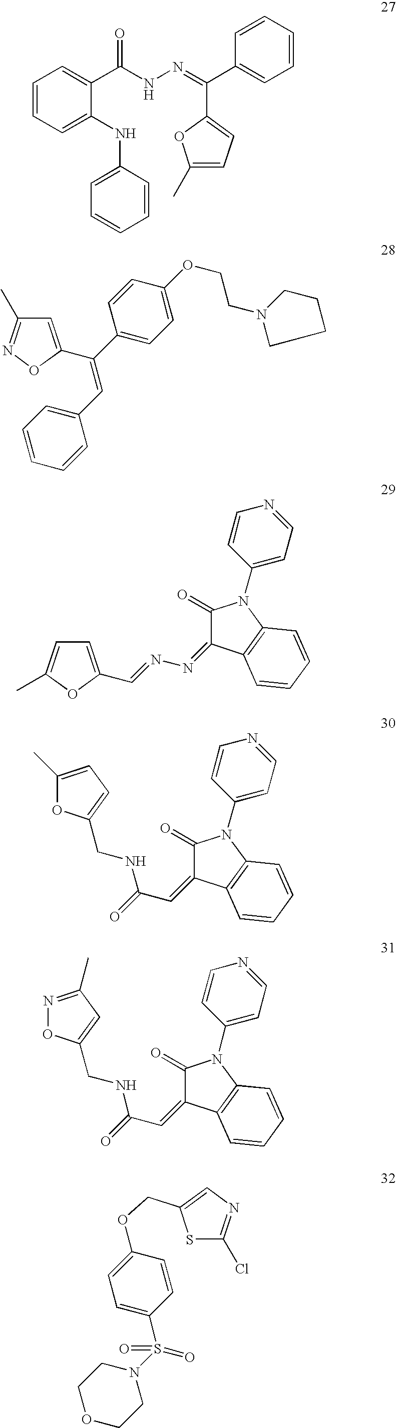

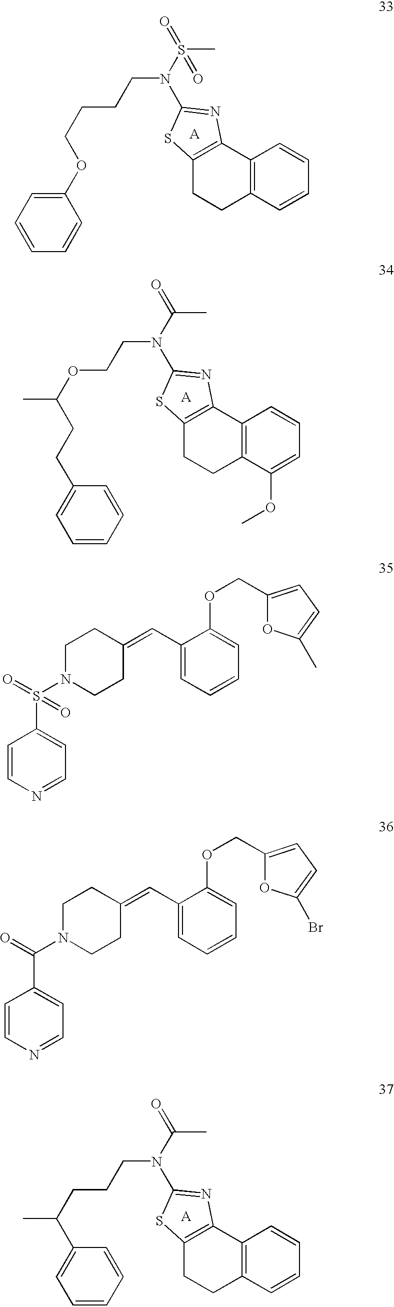

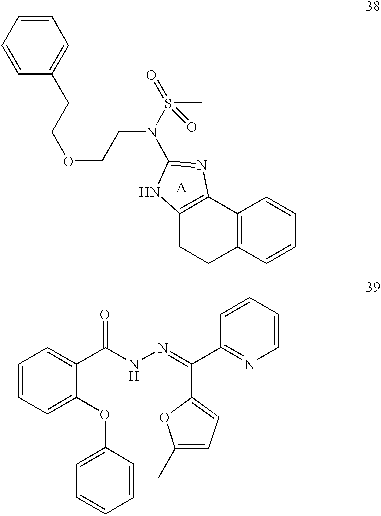

It will be appreciated that in compounds 33, 34 and 37, ring (A), taken together with substituents Z and R, form a 4,5-dihydronaphtho[1,2-d][1,3]thiazol-2-yl ring system, and in compound 38 form a 4,5-dihydro-3H-naphtho[1,2-d]imidazol-2-yl ring system.

1) N′-[(E)-(5-methyl-2-furyl)methylidene]-2-phenoxybenzohydrazide;

2) N′-[(E)-1-(5-methyl-2-thienyl)ethylidene]-2-phenoxyacetohydrazide;

3) 5-[2-(5-methyl-2-furyl)ethyl]-2-(2-thienyl)-1H-indole;

4) 2-(2-furyl)-5-[(E)-2-(5-methyl-2-furyl)ethenyl]-1H-indole;

5) N-[(E)-(5-methyl-2-furyl)methylidene]-4-(4-pyridinyl)-8-quinolinamine;

6) 2-(2-furyl)-5-[2-(5-methyl-2-furyl)ethyl]-1H-indole;

7) 7-{(2E)-2-[(5-methyl-2-furyl)methylene]hydrazino}-N-(2-phenylethyl)-5,6-dihydrobenzo[h]isoquinoline-9-carboxamide;

8) 1-{[(E)-(5-methyl-2-furyl)methylidene]amino}-3-(4-pyridinyl)-2,4(1H,3H)-quinazolinedione;

9) N-(5-methyl-2-furyl)-N-(2′-phenoxy[1,1′-biphenyl]-3-yl)amine;

10) 4-{[7-(5-methyl-2-furyl)-2-naphthyl]oxy}pyridine;

11) N-(5-bromo-1,3,4-oxadiazol-2-yl)-4-hydroxy-2-oxo-6-phenyl-2H-pyran-3-carboxamide;

12) 4-hydroxy-N-(5-methyl-2-furyl)-2-oxo-6-phenyl-2H-pyran-3-carboxamide;

13) 3-[(E)-2-(5-bromo-1,3,4-thiadiazol-2-yl)ethenyl]-4-hydroxy-6-phenyl-2H-pyran-2-one;

14) N-(5-bromo-1,3,4thiadiazol-2-yl)-4-hydroxy-2-oxo-6-phenyl-2H-pyran-3-carboxamide;

15) 5-[(3-amino-1H-1,2,4-triazol-5-yl)methyl]-3-[3-fluoro-4-(4-morpholinyl)phenyl]-1,3-oxazolidin-2-one;

16) 4-[(3-amino-1H-1,2,4-triazol-5-yl)methyl]-1-[3-fluoro-4-(4-morpholinyl)phenyl]-2-imidazolidinone;

17) 1-benzhydryl-4-(5-bromo-2-furoyl)piperazine;

18) 1-benzhydryl-4-[(5-methyl-2-thienyl)carbonyl]piperazine;

19) benzyl (2E)-2-[1-(4-methyl-2-thienyl)ethylidene]hydrazinecarboxylate;

20) 2-(4-chlorophenyl)-6-methyl-5-(5-methyl-1,3,4-oxadiazol-2-yl)[1,3]thiazolo[3,2-b][1,2,4]triazole;

21) N-(5-methyl-3-isoxazolyl)-N′-[(5-phenyl-1,3,4-oxadiazol-2-yl)carbonyl]urea;

22) N-[3-(2-{[(5-chloro-2-thienyl)methyl]sulfonyl}hydrazino)-3-oxopropyl]benzenesulfonamide5-[3-(4-phenoxyphenyl)propyl]-1,3,4oxadiazol-2-ol;

23) N-(3-methyl-5-isoxazolyl)-4-phenoxybenzamide;

24) 4hydroxy-N-(3-methyl-5-isoxazolyl)-2-oxo-6-phenoxy-2H-pyran-3-carboxamide;

25) 2-phenoxy-N′-[(Z)-phenyl(2-thienyl)methylidene]benzohydrazide;

26) 2-anilino-N′-[(Z)-2-furyl(phenyl)methylidene]benzohydrazide;

27) 4-[(Z)-1-(3-methyl-5-isoxazolyl)-2-phenylethenyl]phenyl 2-(1-pyrrolidinyl)ethyl ether;

28) 5-methyl-2-furaldehyde [(3Z)-2-oxo-1-(4-pyridinyl)-1,2-dihydro-3H-indol-3-ylidene]hydrazone;

29) (2Z)-N-[(5-methyl-2-furyl)methyl]-2-[2-oxo-1-(4-pyridinyl)-1,2-dihydro-3H-indol-3-ylidene]ethanamide;

30) (2Z)-N-[(3-methyl-5-isoxazolyl)methyl]-2-[2-oxo-1-(4-pyridinyl)-1,2-dihydro-3H-indol-3-ylidene]ethanamide;

32) (2-chloro-1,3-thiazol-5-yl)methyl 4-(4-morpholinylsulfonyl)phenyl ether;

33) N-(4,5-dihydronaphtho[1,2-d][1,3]thiazol-2-yl)-N-(4-phenoxybutyl)methanesulfonamide;

34) N-(6-methoxy-4,5-dihydronaphtho[1,2-d][1,3]thiazol-2-yl)-N-[2-(1-methyl-3-phenylpropoxy)ethyl]acetamide;

35) 4-{2-[(5-methyl-2-furyl)methoxy]benzylidene}-1-(4-pyridinylsulfonyl)piperidine;

36) 4-{2-[(5-bromo-2-furyl)methoxy]benzylidene}-1-isonicotinoylpiperidine;

37) N-(4,5-dihydronaphtho[1,2-d][1,3]thiazol-2-yl)-N-(4-phenylpentyl)acetamide;

38) N-(4,5-dihydro-3H-naphtho[1,2-d]imidazol-2-yl)-N-[2-(2-phenylethoxy)ethyl]methanesulfonamide;

39) N′-[(Z)-(5-methyl-2-furyl)(2-pyridinyl)methylidene]-2-phenoxybenzohydrazide;

and the pharmaceutically acceptable salts thereof.

The compounds of the invention and the salts thereof can be obtained according to known chemical processes and obvious modifications thereof, well known to the people skilled in the art. For instance compound N′-[(E)-(5-methyl-2-furyl)methylidene]-2-phenoxybenzohydrazide is compound No. 320 (PNU-74654) of WO 87/06127 and it can be obtained as described therein. The preparation of some representative compounds of the invention, is also described in the experimental part of the specification.

The compounds of the invention are active in inhibiting catenin/TCF-4 binding, as proven for instance by the fact that they have been found to be positive in the following tests:

Characterization of the Binding by ITC

Compounds selected from docking studies obtained from commercially available programs were screened in TCF-4 competition assays using Isothermal Titration μ-Calorimetry (ITC). The difference in binding affinity of TCF-4 (residues 1-56) to β-catenin/armadillo was determined in the presence of a total inhibitor concentration of 50 μM. The compounds were screened as mixtures of four compounds in each titration experiment. Compound mixtures that showed at least a 3-fold reduction in TCF-4 binding affinity were selected for further characterization. β-Catenin binders in the screened mixtures were identified either prior or after ITC competition assays by NMR. Direct ITC binding assays were used to determine binding constants for the identified TCF-4 competitive inhibitors.

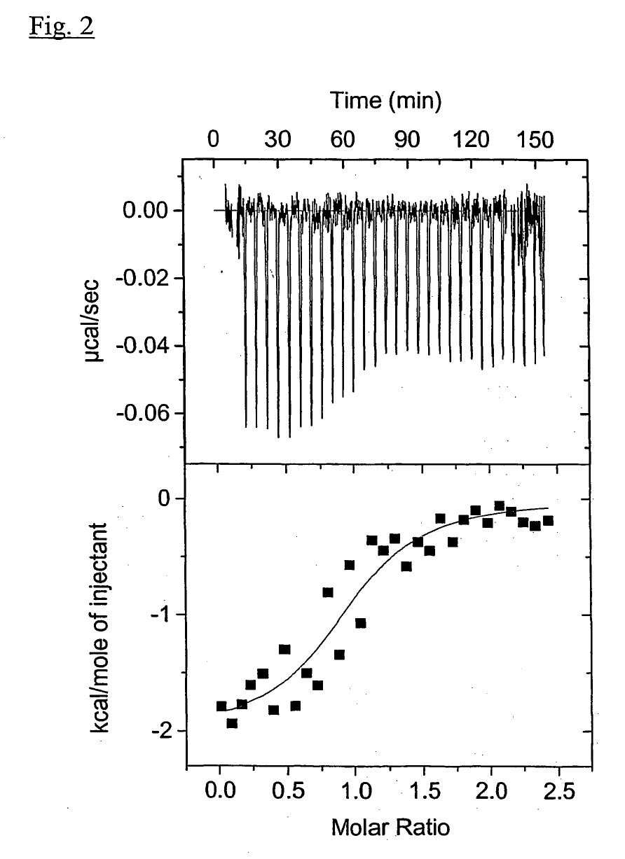

For example, the compound of the invention PNU-74654 has been identified to bind strongly to β-catenin with the following thermodynamic binding characteristics: K_{B}: 2.2±0.9 10⁶ Mol⁻¹, (K_{D} 450 nM), ΔH: −2.0±0.5 kcal/mol and stoichiometry of 1:1 (FIG. 2). This compound reduced TCF-4 affinity for β-Catenin about 10-fold.

FIG. 2 shows the experimental calorimetric data of the binding of compound PNU074654 to the armadillo repeat region of β-Catenin. Titrations were performed at 20° C. using a buffer containing PBS (Sigma) with 1 mM DTT. PNU074654 was titrated of into β-Catenin/armadillo. The top panel shows the raw heat data obtained over a series of injections of PNU074654 β-catenin/armadillo (5 μM). The integrated heat signals of the data shown in the top panel of the figure gave rise to the binding curve shown in the lower panel. The solid line represents a calculated curve using the best-fit parameters obtained by a nonlinear least-squares fit.

NMR Screening

The WaterLOGSY NMR screening method developed in our laboratories (C. Dalvit, P. Pevarello, M. Tatò, M. Veronesi, A. Vulpetti and M. Sundström: “Identification of compounds with binding affinity to proteins via magnetization transfer from bulk water” Journal of Biomolecular NMR, 18 65-68, 2000) has been validated as a highly sensitive tool for identifying binders to various targets. The method exploits the transfer of bulk water magnetization through different relay pathways to the small molecule interacting with the receptor. The method is particularly suited for the identification of protein-protein interaction antagonists.

Material and Methods

The protein concentration used for the WaterLOGSY experiments was 2 μM in 5 mM Tris, 10 mM NaCl pH 7.3. Compounds were screened at 20° C. first in mixtures at a 50 μM concentration. Compounds that were identified to bind to β-catenin/armadillo were verified using the individual compounds.

NMR WaterLOGSY competition binding studies were then performed in order to differentiate between binders and true antagonists. The concentration of β-catenin armadillo repeat units, TCF-4 and ligand was 2, 25 and 50 μM, respectively.

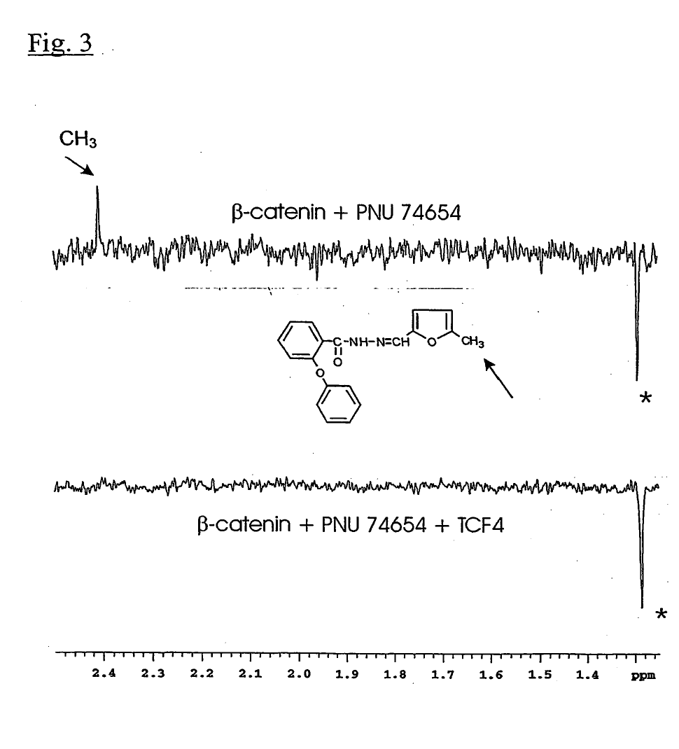

For instance, compound PNU-74654 could-be verified as a protein-protein interaction antagonist (see FIG. 3). The NMR spectra for the protein solutions with and without TCF-4 were recorded with 2048 and 800 scans, respectively. A larger number of scans were recorded for the solution in the presence of TCF-4 in order to detect the complete displacement of PNU 74654 from β-catenin.

As can be seen from FIG. 3, the methyl group resonance of the compound (indicated by an arrow) appears as a positive signal in the WaterLOGSY spectrum of the β-catenin+PNU 74654 solution. This is a clear indication that PNU 74654 is a binder to this target. The resonance is missing in the spectrum recorded for the same solution in the presence of TCF-4 (lower spectrum). These data further support that the compound is an antagonist of the β-catenin-TCF-4 interaction. The asterisk indicates the resonance of a compound (impurity) that does not interact with the protein.

In view of the above the compounds of the invention are useful as TCF-4/β-catenin interaction modulating compounds, in particular as interaction inhibitors, and thus in preventing and treating proliferative disorders, in particular cancer, in FAP patients, patients with APC or β-catenin mutations or patients with increased risk of developing cancer, in inhibiting cancer metastasis, in treating Alzheimer's disease and in modulating hair growth. Examples of cancers that can be prevented and treated by the compounds of the invention are colorectal carcinoma, melanoma, liver carcinoma, breast cancer and prostate cancer.

A compound of the invention can be administered to a mammal, including humans, through any administration route, the oral and parenteral ones being the preferred. The compounds are preferably administered in the form of a suitable pharmaceutical form, as known to the people skilled in the art. Suitable dosages for a compound of the invention for an adult human may range from about 1 mg to about 500 mg pro dose, from 1 to 5 times daily.

The following preparations and formulation examples are representative of the present invention.

### PREPARATION EXAMPLES

### EXAMPLE 1

### 1 Benzidryl-4-(5-bromo-2-furoyl)piperazine (17)

A solution of 5-bromo-2-furoylchloride (g 0.45) in pyridine (ml 5) was added dropwise to a stirred solution of 1-benzidrylpiperazine (g 0.6) in pyridine (ml 10). After stirring overnight at room temperature, the solvent was removed in vacuo and the residue taken up in ethylacetate was washed with brine and dried. The solvent was removed and the residue was filtered of a small pad of silica gel eluting with ethylacetate to give after crystallization from ethylacetate, the title compound (g 0.65) in 71% yield.

### EXAMPLE 2

### 1-Benzidryl-4-[(5-methyl-2-thienyl)carbonyl]piperazine (18)

Operating as Example 1, but employing 5-methyl-2-thienylchloride instead of 5-bromo-2-furoylchloride, the title compound was obtained in 47% yield.

### EXAMPLE 3

### N-(5-Methyl-3-isoxazolyl)-N′-[(5-phenyl-1,3,4-oxadiazol-2-yl)carbonyl]urea (21)

A stirred solution of 5 phenyl-1,3,4-oxadiazol-2-carboxamide (g 2) and 5-methyl-isoxazol-3-isocyanate (g 3.7) in dioxane (ml 35) was refluxed for 3 days. The solvent was removed and the residue was chromatographed on silica gel eluting with ethylacetate/cyclohexane 1/1, to provide after crystallization from acetone, the title compound (g 0.35) in 8% yield.

### EXAMPLE 4

### N-(3-Methyl-5-isoxazolyl)-4phenoxybenzamide (24)

Operating as in Example 1, but employing 4-phenoxybenzoylchloride instead of 5-bromo-2-furoylchloride and 3-methyl-5-amino-isoxazole instead of 1-benzidrylpiperazine, the title compound was obtained in 57% yield.

### EXAMPLE 5

### (2-Chloro-1,3-thiazol-5-yl)methyl 4-(4-morpholinylsulfonyl ether (32)

To stirred solution of 4-hydroxy-morpholinbenzensolphonamide (g 2.4) in DMF (ml 35) was added portionwise 60% sodium hydride (g 0.41) at room temperature. After stirring for 1 h, 2-chloro-5-chlormethyl-thiazole (g 1.6) at room temperature. After stirring overnight, the solution was diluted with ethylacetate and thoroughly washed with brine and dried. The residue was filtered on a small pad of silica gel to provide the title compound (g 2.1) in 67% yield.

### EXAMPLE 6

### 4-{2-[(5-Methyl-2-furyl)methoxy]benzylidene}-1-(4-pyridinylsulfonyl)piperidine(35)

To a stirred solution of 4-piperidone (g 5) in pyridine (ml 30) was added dropwise a solution of 4-pyridinsolphonylchloride hydrochloride (g 12) in pyridine (ml 50). After stirring for 5 hours at room temperature, the solvent was removed and the residue taken up in ethylacetate was thoroughly washed with 0.1 M Na2CO3 then with brine and dried. Concentration of the solution to small volume afforded 4-(4-pyridinylsolphonyl)-4-piperidone (g 7.4).

To a stirred solution of {2-[5-methyl-2-furyl)methoxy]benzylydene}(triphenyl)phosphorane (g 5) in THF (ml 75) was added dropwise a solution of 4-(4-pyridinylsolphonyl)-4-piperidone (g 2.9) in THF (ml 75) at −10° C. After stirring for 1 h at −10° C., the yellowish solution was set aside overnight at room temperature. The solvent was removed and the residue dissolved in ethylacetate was washed with brine then dried. The crude reaction mixture was carefully chromatographed on silica gel eluting with cyclohexane/ethylacetate 3/1 to provide after crystallization from a small volume of ethanol, the title compound (g 1.3) in 27% yield.

### EXAMPLE 7

### 4-{2-[(5-Bromo-2-furyl)methoxy]benzylidene}-1-isonicotinoylpiperidine (36)

Operating as in Example 5, but employing isonicotinoylchloride hydrochloride instead of 4-pyridinsolphonylchloride hydrochoride and {2-[5-bromo-2-furyl)methoxy]benzylydene}(triphenyl)phosphorane instead of {2-[5-methyl-2-furyl)methoxy]benzylydene}(triphenyl)phosphorane, the title compound was obtained in 32% yield.

### EXAMPLE 8

### N-(6-Methoxy-4,5-dihydronaphtho[1,2-d][1,3]thiazol-2-yl)-N-[2-(1-methyl-3-phenylpropoxy)ethyl]acetamide (34)

A stirred suspension of (2-amino-6-methoxy)-4,5-dihydronaphtho[1,2-d][1,3]thiazole (g 3.2) and [3-(2-bromoethoxy)butyl]benzene (g 3.8) and potassium carbonate (g 2) in DMF (ml 55) was heated at 65° C. for 5 h. The solvent was removed and the residue partitioned between ethylacetate and brine. After drying, the solvent was removed and the crude product was filtered on a small pad of silica gel eluting with ethylacetate/cyclohexane 3/2 to give after crystallization from ethanol, (6-methoxy-4,5-dihydronaphtho[1,2-d][1,3]thiazol-2-yl)-N-[2-(1-methyl-3-phenylpropoxy)ethyl] (g in 42% yield.

To a solution of (6-methoxy-4,5-dihydronaphtho[1,2-d][1,3]thiazol-2-yl)-N-[2-(1-methyl-3-phenylpropoxy)ethyl] (g 1) in pyridine (ml 15) was added acetic anhydride (ml 0.5) at room temperature. The solution was set aside for 3 h, then diluted with ethylacetate and washed with 0.1 M HCl, then with brine and dried. The residue was twice crystallized from acetone to furnish the title compound (g 0.8) in 78% yield.

### EXAMPLE 9

### N-(4,5-Dihydronaphtho[1,2-d][1,3]thiazol-2-yl)-N-(4-phenylpentyl)acetamide (37)

Operating as in Example 8, but employing of (2-amino)-4,5-dihydronaphtho[1,2-d][1,3]thiazole instead of (2-amino-6-methoxy)-4,5-dihydronaphtho[1,2-d][1,3]thiazole and (4bromo-1-methylbutyl)benzene instead of [3-(2-bromoethoxy)butyl]benzene, the title compound was obtained in 19% yield.

### EXAMPLE 10

### N-(4,5-Dihydro-3H-naphtho[1,2-d]imidazol-2-yl)-N-[2-(2-phenylethoxy)ethyl]methanesulfonamide (38)

Operating as in Example 8, but employing of (2-amino)-4,5-dihydronaphtho[1,2-d][1,3]imidazole instead of (2-amino-6-methoxy)-4,5-dihydronaphtho[1,2-d][1,3]thiazole and [2-(2-bromoethoxy)ethyl]benzene instead of [3-(2-bromoethoxy)butyl]benzene and mesylchloride instead of acetic anhydride, the title compound was obtained in 12% overall yield.

### FORMULATION EXAMPLES

### EXAMPLE 1

### Dry Capsules

5000 capsules, each of which contain 0.25 g of one of the compounds of the formula (I) mentioned in the preceding Examples as active ingredient, are prepared as follows:

Composition Active ingredient 1250 g

Talc 180 g

Wheat starch 120 g

Magnesium stearate 80 g

Lactose 20 g

Preparation process: The powdered substances mentioned are pressed through a sieve of mesh width 0.6 mm. Portions of 0.33 g of the mixture are transferred to gelatine capsules with the aid of a capsule-filling machine.

### EXAMPLE 2

### Soft Capsules

5000 soft gelatine capsules, each of which contain 0.05 g of one of the compounds of the formula(I) mentioned in the preceding Examples as active ingredient, are prepared as follows:

Composition Active ingredient 250 g

Lauroglycol 2 litres

Preparation process: The powdered active ingredient is suspended in Lauroglykole (propylene glycol laurate, Gattefoss S. A., Saint Priest, France) and ground in a wet-pulveriser to a particle size of about 1 to 3 gm. Portions of in each case 0.419 g of the mixture are then transferred to soft gelatine capsules by means of a capsule-filling machine.

### EXAMPLE 3

### Soft Capsules

5000 soft gelatine capsules, each of which contain 0.05 g of one of the compounds of the formula (I) mentioned in the preceding or following Examples as active ingredient, are prepared as follows:

Composition Active ingredient 250 g

PEG 400 1 litre

Tween 80 1 litre

Preparation process: The powdered active ingredient is suspended in PEG 400 (polyethylene glycol of Mr between 380 and about 420, Sigma, Fluka, Aldrich, USA) and Tween' 80 (polyoxyethylene sorbitan monolaurate, Atlas Chem. Inc., USA, supplied by Sigma, Fluka, Aldrich) and ground in a wet-pulveriser to a particle size of about 1 to 3 mm. Portions of in each case 0.43 g of the mixture are then transferred to soft gelatine capsules by means of a capsule-filling machine.

1. Pharmacophore (IA), characterized by a structure which comprises:
a saturated, partially saturated, carbocyclic or heteroaromatic pentatomic ring (A), substituted at least by a substituent (Z) pharmacophore (IA), characterized by a structure which comprises: a saturated, partially saturated, carbocyclic or heteroaromatic pentatomic ring (A), substituted at least by a substituent (Z) selected independently from hydrogen, halogen, hydroxy, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl; or (Z) and R, taken together, form an optionally substituted, partially saturated monocyclic or bicyclic ring system;
an optionally substituted, saturated, partially saturated, carbocyclic, aromatic or internally condensed ring (B); rings (A) and (B) being separated by a spacer (Y) which provides an inter-center distance between rings (A) and (B) of about 10.9±2 Angstrom; wherein the relative orientation between said rings (A) and (B) is such that the angle θ between the two centroid vectors is about 40 degrees ±30 degrees; the convention for the orientation of the vectors being such that cos θ is >0.

2. A screening method for identifying a candidate drug for use in Familial Adenomatous Polyposis (FAP) patients, patients with APC or β-catenin mutations, or patients with increased risk of developing cancer, comprising the steps of determining the optimal fit of a plurality of compounds into pharmacophore (IA), as defined in claim 1, such that the lowest energy of interaction and the best steric fit are obtained.

3. Use of a compound as identified by the screening method of claim 2 in the preparation of a medicament which is able to interact with β-catenin/TCF-4 binding site.

4. A β-catenin/TCF-4 interaction modulating compound capable of adopting a structure having a pharmacophoric pattern essentially equivalent to the pharmacophoric pattern of pharmacophore (IA), as defined in claim 1.

5. The use of a compound of formula (I) or a pharmaceutically acceptable salt thereof, having the following formula: 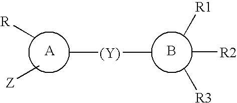
wherein:
(A) is a saturated, partially saturated, carbocyclic or heteroaromatic pentatomic ring;
(B) is a saturated, partially saturated, carbocyclic, aromatic or internally condensed ring;
(Y), in its shortest way, is a spacer consisting of about 4 to 9 chain atoms chosen independently from C, O, N and S, which may have independently different hybridization states, and wherein two to five adjacent atoms of the chain my be part of an optionally substituted aryl, heteroaryl or partially saturated aryl or heteroaryl ring system, which may be either isolated or include ring (B).
Z is a substituent selected independently from hydrogen, halogen, hydroxy, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl;
R is independently selected from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl; or Z and R, taken together, form an optionally substituted, partially saturated monocyclic or bicyclic ring system; or Z and R, taken together, form an optionally substituted, partially saturated monocyclic or bicyclic ring system;
each of R1, R2 and R3, which may be independently the same or different, is chosen from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl; a NHCORc or NHSO2Rc group wherein Rc is C1-C4 alkyl; and a C5-C6 cycloalkyl-oxy or aryloxy group, in the preparation of a pharmaceutical composition, for use in inhibiting β-catenin/TCF-4 interaction.

6. The use according to claim 5, wherein spacer (Y) is selected from:

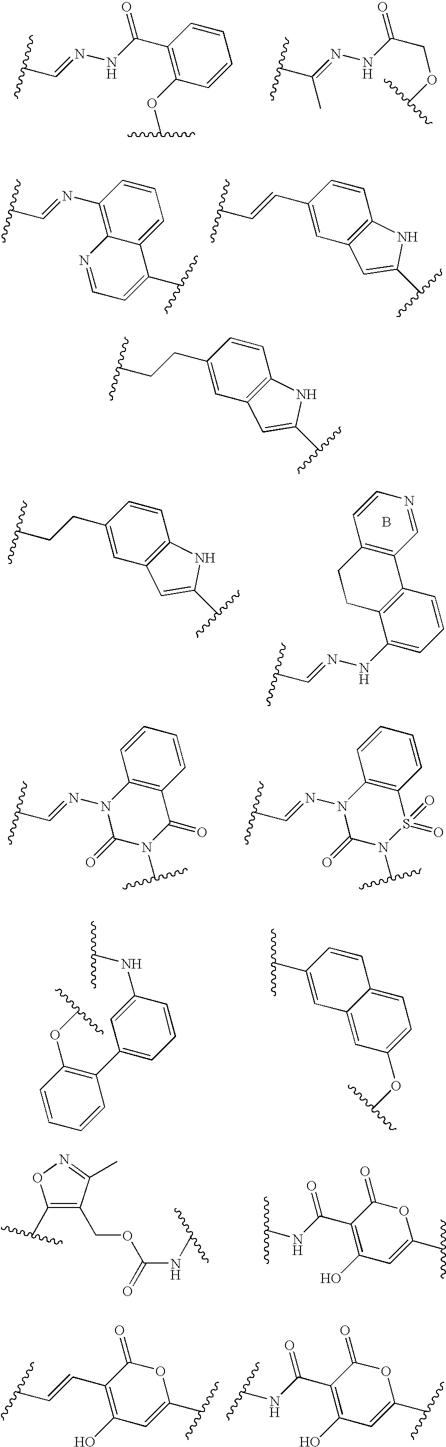

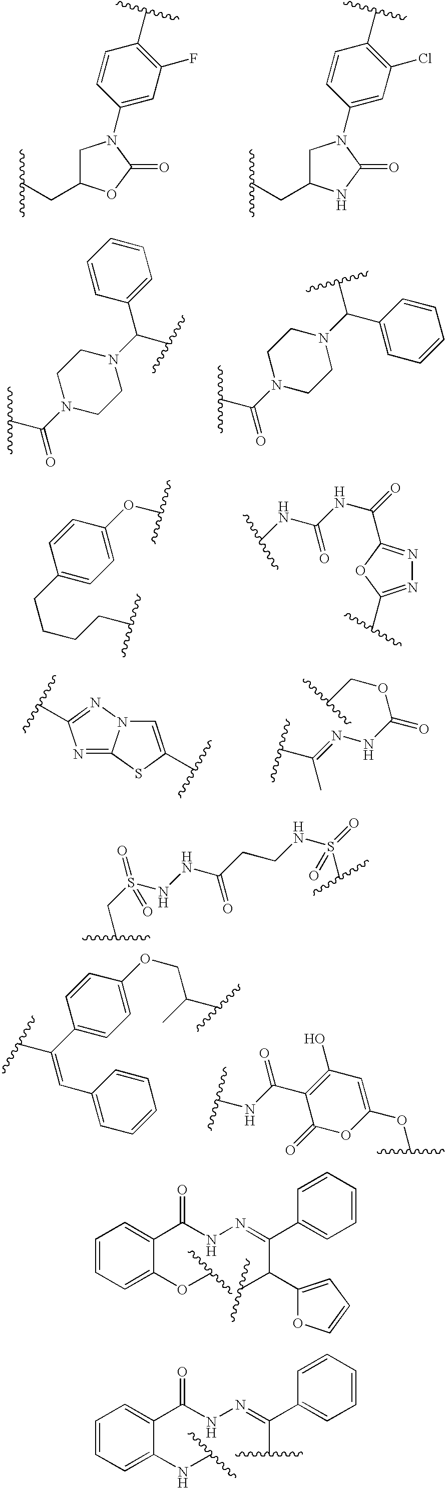

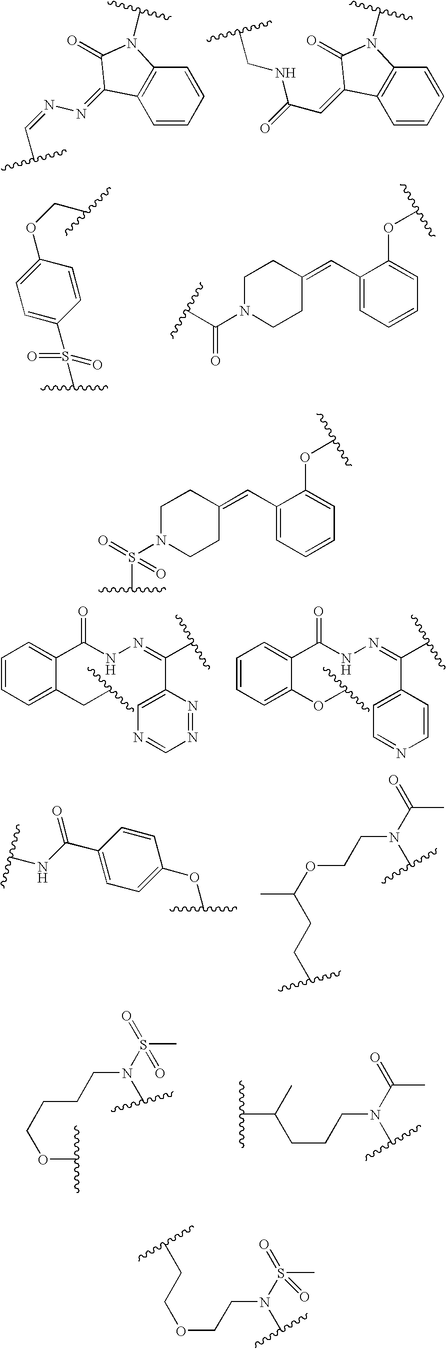

7. The use according to claim 5, wherein in the compound of formula (I)
(A) is a ring selected from cyclopentyl, pyrrolidine, furane, pyrrole, thiophene, oxazole, isoxazole, imidazole, thiazole, oxadiazole, thiadiazole and triazole.
(B) is a ring selected from cyclopentyl, cyclohexyl, cycloheptyl, pyrrolidine, piperazine, piperidine, morpholino, hexahydroazepine, cyclohexene, piperideino, tetrahydroquinoline, tetrahydroisoquinoline, dihydropyrrole, phenyl, naphthyl, furane, pyrrole, thiophene, oxazole, isoxazole, imidazole, thiazole, oxadiazole, thiadiazole, triazole, pyridine, pyrimidine, pyridazine, pyrazine, quinoline, isoquinoline, benzothiazole, benzoimidazole and benzoxazole;
spacer (Y) is selected from 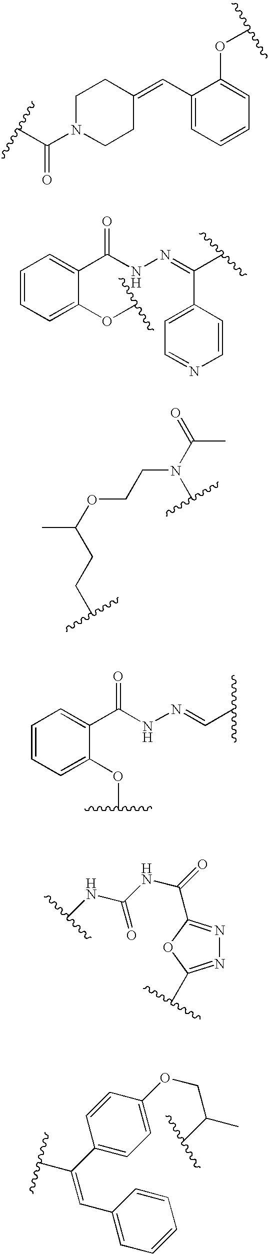
Z is a substituent selected from hydrogen, halogen, hydroxy, cyano, C1-C4 alkyl, trifluoromethyl, C1-C4 alkoxy, amino, methylamino, ethylamino, dimethylamino, diethylamino, NHCOC2H5 and NHSO2CH3.
R is from hydrogen, halogen, cyano, C1-C4 alkyl, trifluoromethyl, C1-C4 alkoxy, amino, methylamino, ethylamino, dimethylamino, diethylamino, NHCOC2H5 and NHSO2CH3; or Z and R, taken together, form a partially saturated phenyl or naphthalene ring;
each of R1, R2 and R3 is independently chosen from hydrogen, halogen, cyano, C1-C4 alkyl, trifluoromethyl, C1-C4 alkoxy, amino, methylamino, ethylamino, dimethylamino, diethylamino, NHCOC2H5, NHSO2CH3, cyclopentyloxy and cyclohexyl.

8. The use according to claim 5, wherein in the compound of formula (I)
(A) is a ring selected from furane, thiadiazole, isoxazole, thiophene, pyrrolidine, triazole, oxadiazole and thiazole;
(B) is a ring selected from furane, pyridine, phenyl, morpholine, isoxazole, pyrrolidine and thiazole;
spacer (Y) is selected from 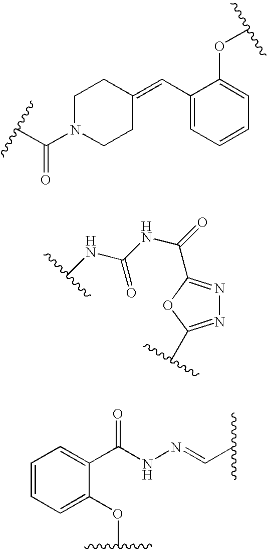
substituent (Z) is hydrogen, halogen, amino, hydroxy, C1-C4 alkyl and C1-C4 alkoxy,
R is hydrogen; or Z and R, taken together with ring (A) form a 4,5-dihydronaphtho[1,2-d][1,3]thiazol-2-yl or 4,5-dihydro-3H-naphtho[1,2-d]imidazol-2-yl ring system;
each of R1, R2 and R3 is independently chosen from hydrogen, amino, hydroxy, C1-C4 alkyl and C1-C4 alkoxy.

9. The use according to claim 5, wherein the compound of formula (I) is selected from:
1) N′-[(E)-(5-methyl-2-furyl)methylidene]-2-phenoxybenzohydrazide;
2) N′-[(E)-1-(5-methyl-2-thienyl)ethylidene]-2-phenoxyacetohydrazide;
3) 5-[2-(5-methyl-2-furyl)ethyl]-2-(2-thienyl)-1H-indole;
4) 2-(2-furyl)-5-[(E)-2-(5-methyl-2-furyl)ethenyl]-1H-indole;
5) N-[(E)-(5-methyl-2-furyl)methylidene]-4-(4-pyridinyl)-8-quinolinamine;
6) 2-(2-furyl)-5-[2-(5-methyl-2-furyl)ethyl]-1H-indole;
7) 7-{(2E)-2-[(5-methyl-2-furyl)methylene]hydrazino}-N-(2-phenylethyl)-5,6-dihydrobenzo[h]isoquinoline-9-carboxamide;
8) 1-{[(E)-(5-methyl-2-furyl)methylidene]amino}-3-(4-pyridinyl)-2,4(1H,3H)-quinazolinedione;
9) N-(5-methyl-2-furyl)-N-(2′-phenoxy[1,1′-biphenyl]-3-yl)amine;
10) 4-{[7-(5-methyl-2-furyl)-2-naphthyl]oxy}pyridine;
11) N-(5-bromo-1,3,4-oxadiazol-2-yl)-4-hydroxy-2-oxo-6-phenyl-2H-pyran-3-carboxamide;
12) 4-hydroxy-N-(5-methyl-2-furyl)-2-oxo-6-phenyl-2H-pyran-3-carboxamide;
13) 3-[(E)-2-(5-bromo-1,3,4-thiadiazol-2-yl)ethenyl]-4-hydroxy-6-phenyl-2H-pyran-2-one;
14) N-(5-bromo-1,3,4-thiadiazol-2-yl)-4-hydroxy-2-oxo-6-phenyl-2H-pyran-3-carboxamide;
15) 5-[(3-amino-1H-1,2,4-triazol-5-yl)methyl]-3-[3-fluoro-4-(4-morpholinyl)phenyl]-1,3-oxazolidin-2-one;
16) 4-[(3-amino-1H-1,2,4-triazol-5-yl)methyl]-1-[3-fluoro-4-(4-morpholinyl)phenyl]-2-imidazolidinone;
17) 1-benzhydryl-4-(5-bromo-2-furoyl)piperazine;
18) 1-benzhydryl-4-[(5-methyl-2-thienyl)carbonyl]piperazine;
31) benzyl (2E)-2-[1-(4-methyl-2-thienyl)ethylidene]hydrazinecarboxylate;
32) 2-(4-chlorophenyl)-6-methyl-5-(5-methyl-1,3,4-oxadiazol-2-yl)[1,3]thiazolo[3,2-b][1,2,4]triazole;
33) N-(5-methyl-3-isoxazolyl)-N′-[(5-phenyl-1,3,4-oxadiazol-2-yl)carbonyl]urea;
34) N-[3-(2-{[(5-chloro-2-thienyl)methyl]sulfonyl}hydrazino)-3-oxopropyl]benzenesulfonamide5-[3-(4-phenoxyphenyl)propyl]-1,3,4-oxadiazol-2-ol;
35) N-(3-methyl-5-isoxazolyl)-4-phenoxybenzamide;
36) 4-hydroxy-N-(3-methyl-5-isoxazolyl)-2-oxo-6-phenoxy-2H-pyran-3-carboxamide;
37) 2-phenoxy-N′-[(Z)-phenyl(2-thienyl)methylidene]benzohydrazide;
38) 2-anilino-N′-[(Z)-2-furyl(phenyl)methylidene]benzohydrazide;
39) 4-[(Z)-1-(3-methyl-5-isoxazolyl)-2-phenylethenyl]phenyl 2-(1-pyrrolidinyl)ethyl ether;
40) 5-methyl-2-furaldehyde [(3Z)-2-oxo-1-(4-pyridinyl)-1,2-dihydro-3H-indol-3-ylidene]hydrazone;
41) (2Z)-N-[(5-methyl-2-furyl)methyl]-2-[2-oxo-1-(4-pyridinyl)-1,2-dihydro-3H-indol-3-ylidene]ethanamide;
42) (2Z)-N-[(3-methyl-5-isoxazolyl)methyl]-2-[2-oxo-1-(4-pyridinyl)-1,2-dihydro-3H-indol-3-ylidene]ethanamide;
32) (2-chloro-1,3-thiazol-5-yl)methyl 4-(4-morpholinylsulfonyl)phenyl ether;
33) N-(4,5-dihydronaphtho[1,2-d][1,3]thiazol-2-yl)-N-(4-phenoxybutyl)methanesulfonamide;
34) N-(6-methoxy-4,5-dihydronaphtho[1,2-d][1,3]thiazol-2-yl)-N-[2-(1-methyl-3-phenylpropoxy)ethyl]acetamide;
35) 4-{2-[(5-methyl-2-furyl)methoxy]benzylidene}-1-(4-pyridinylsulfonyl)piperidine;
36) 4-{2-[(5-bromo-2-furyl)methoxy]benzylidene}-1-isonicotinoylpiperidine;
37) N-(4,5-dihydronaphtho[1,2-d][1,3]thiazol-2-yl)-N-(4-phenylpentyl)acetamide;
38) N-(4,5-dihydro-3H-naphtho[1,2-d]imidazol-2-yl)-N-[2-(2-phenylethoxy)ethyl]methanesulfonamide;
39) N′-[(Z)-(5-methyl-2-furyl)(2-pyridinyl)methylidene]-2-phenoxybenzohydrazide;
or a pharmaceutically acceptable salt thereof.

10. The use according to claim 5, wherein the medicament is for use in preventing and treating proliferative disorders, including cancer, in inhibiting cancer metastasis, in treating Alzheimer's disease and in modulating hair growth.

11. The use according to claim 5, wherein the medicament is for use in preventing and treating colorectal carcinoma, melanoma, liver carcinoma, breast cancer and prostatic cancer.

12. A compound of formula (I) or a pharmaceutically acceptable salt thereof, as defined in claim 5, for use as a medicament, provided that such compound is other than N′-[(E)-(5-methyl-2-furyl)methylidene]-2-phenoxybenzohydrazide.

13. A compound according to claim 12, for use in inhibiting β-catenin/TCF-4 interaction.

14. A compound according to claim 12, for use in preventing and treating proliferative disorders, including cancer, in inhibiting cancer metastasis, in treating Alzheimer's disease and in modulating hair growth.

15. A compound according to claim 12, for use in preventing and treating colorectal carcinoma, melanoma, liver carcinoma, breast cancer and prostatic cancer.

16. A compound of formula (I) or a pharmaceutically acceptable salt thereof, having the following formula 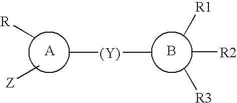
wherein:
(A) is a saturated, partially saturated, carbocyclic or heteroaromatic pentatomic ring;
(B) is a saturated, partially saturated, carbocyclic, aromatic or internally condensed ring;
(Y), in its shortest way, is a spacer consisting of about 4 to 9 chain atoms chosen independently from C, O, N and S, which may have independently different hybridization states, and wherein two to five adjacent atoms of the chain my be part of an optionally substituted aryl, heteroaryl or partially saturated aryl or heteroaryl ring system, which may be either isolated or include ring (B).
Z is a substituent selected independently from hydrogen, halogen, hydroxy, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl;
R is independently selected from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl; or Z and R, taken together, form an optionally substituted, partially saturated monocyclic or bicyclic ring system; or Z and R, taken together, form an optionally substituted, partially saturated monocyclic or bicyclic ring system;
each of R1, R2 and R3, which may be independently the same or different, is chosen from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl; a NHCORc or NHSO2Rc group wherein Rc is C1-C4 alkyl; and a C5-C6 cycloalkyl-oxy or aryloxy group, provided that such compound is other than N′-[(E)-(5-methyl-2-furyl)methylidene]-2-phenoxybenzohydrazide.

17. A compound of formula (I), according to claim 16, wherein spacer (Y) is selected from:

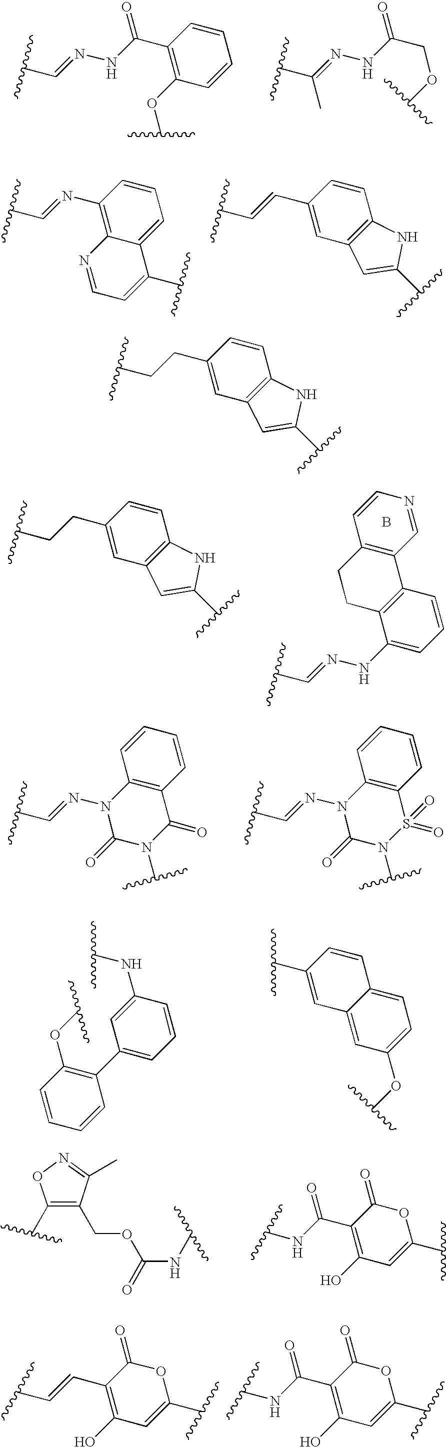

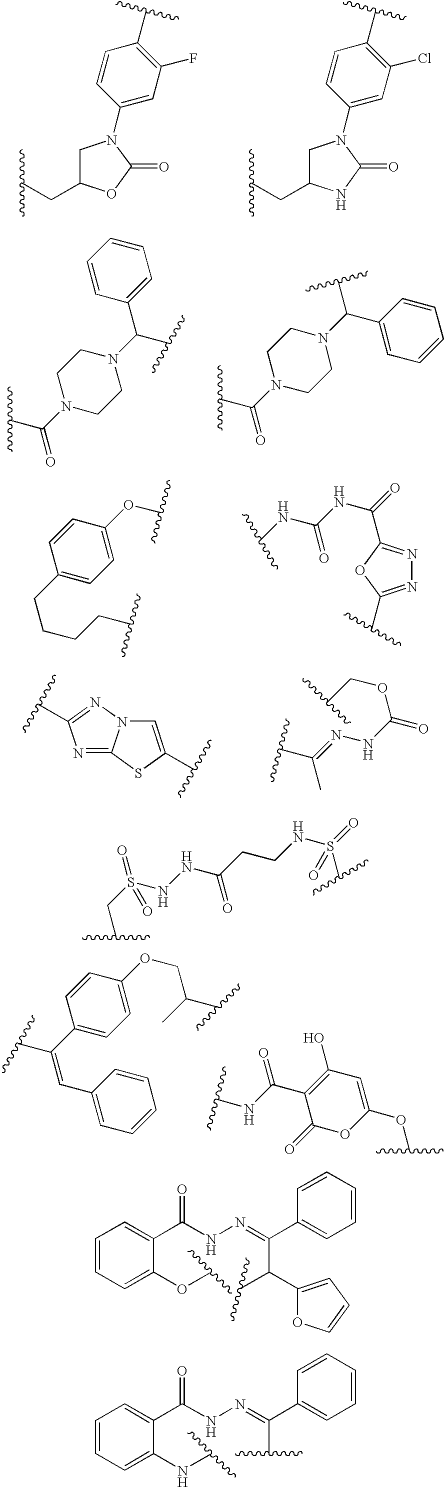

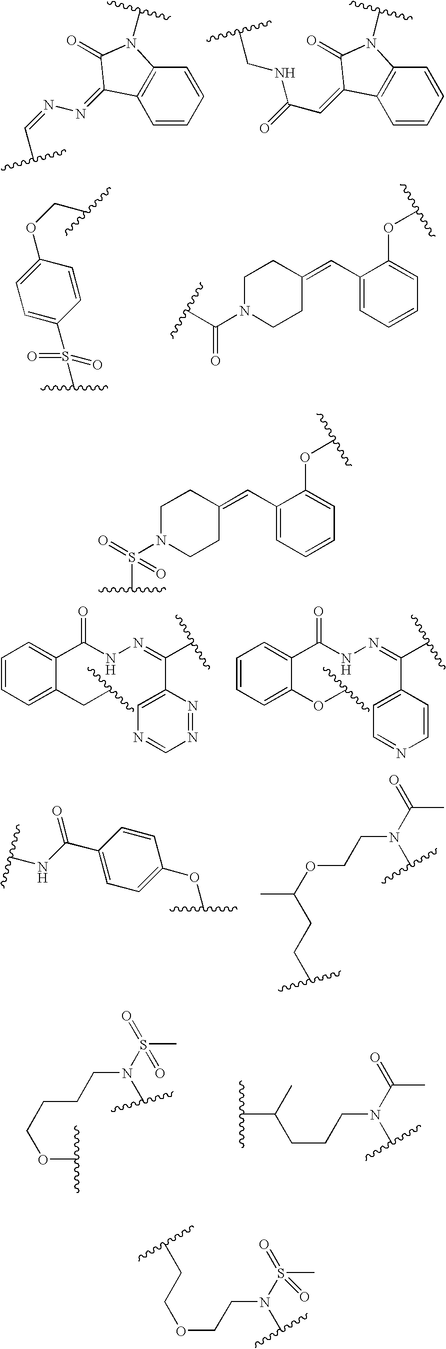

18. A compound of formula (I) according to claim 16, wherein
(A) is a ring selected, from cyclopentyl, pyrrolidine, furane, pyrrole, thiophene, oxazole, isoxazole, imidazole, thiazole, oxadiazole, thiadiazole and triazole.
(B) is a ring selected from cyclopentyl, cyclohexyl, cycloheptyl, pyrrolidine, piperazine, piperidine, morpholino, hexahydroazepine, cyclohexene, piperideino, tetrahydroquinoline, tetrahydroisoquinoline, dihydropyrrole, phenyl, naphthyl, furane, pyrrole, thiophene, oxazole, isoxazole, imidazole, thiazole, oxadiazole, thiadiazole, triazole, pyridine, pyrimidine, pyridazine, pyrazine, quinoline, isoquinoline, benzothiazole, benzoimidazole and benzoxazole;
spacer (Y) is selected from 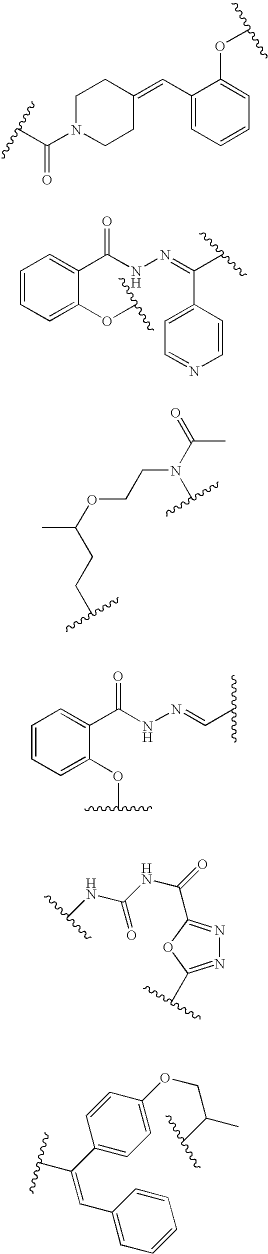
Z is a substituent selected from hydrogen, halogen, hydroxy, cyano, C1-C4 alkyl, trifluoromethyl, C1-C4 alkoxy, amino, methylamino, ethylamino, dimethylamino, diethylamino, NHCOC2H5 and NHSO2CH3.
R is from hydrogen, halogen, cyano, C1-C4 alkyl, trifluoromethyl, C1-C4 alkoxy, amino, methylamino, ethylamino, dimethylamino, diethylamino, NHCOC2H5 and NHSO2CH3; or Z and R, taken together, form a partially saturated phenyl or naphthalene ring;
each of R1, R2 and R3 is independently chosen from hydrogen, halogen, cyano, C1-C4 alkyl, trifluoromethyl, C1-C4 alkoxy, amino, methylamino, ethylamino, dimethylamino, diethylamino, NHCO-ethyl, NHSO2-methyl, cyclopentyloxy and cyclohehyloxy.

19. A compound of formula (I) according to claim 16, wherein
(A) is a ring selected from furane, thiadiazole, isoxazole, thiophene, pyrrolidine, triazole, oxadiazole and thiazole;
(B) is a ring selected from furane, pyridine, phenyl, morpholine, isoxazole, pyrrolidine and thiazole;
spacer (Y) is selected from 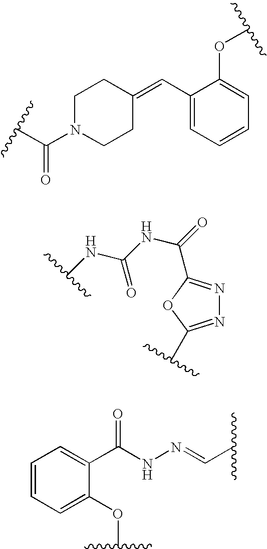
substituent (Z) is hydrogen, halogen, amino, hydroxy, C1-C4 alkyl and C1-C4 alkoxy;
R is hydrogen; or Z and R, taken together with ring (A) form a 4,5-dihydronaphtho[1,2-d][1,3]thiazol-2-yl or 4,5-dihydro-3H-naphtho[1,2-d]imidazol-2-yl ring system;
each of R1, R2 and R3 is independently chosen from hydrogen, amino, hydroxy, C1-C4 alkyl and C1-C4 alkoxy.

20. A compound of formula (I) according to claim 16, selected from:
2) N′-[(E)-1-(5-methyl-2-thienyl)ethylidene]-2-phenoxyacetohydrazide;
3) 5-[2-(5-methyl-2-furyl)ethyl]-2-(2-thienyl)-1H-indole;
4) 2-(2-furyl)-5-[(E)-2-(5-methyl-2-furyl)ethenyl]-1H-indole;
5) N-[(E)-(5-methyl-2-furyl)methylidene]-4-(4-pyridinyl)-8-quinolinamine;
6) 2-(2-furyl)-5-[2-(5-methyl-2-furyl)ethyl]-1H-indole;
7) 7-{(2E)-2-[(5-methyl-2-furyl)methylene]hydrazino}-N-(2-phenylethyl)-5,6-dihydrobenzo[h]isoquinoline-9-carboxamide;
8) 1-{[(E)-(5-methyl-2-furyl)methylidene]amino}-3-(4-pyridinyl)-2,4(1H,3H)-quinazolinedione;
9) N-(5-methyl-2-furyl)-N-(2′-phenoxy[1,1′-biphenyl]-3-yl)amine;
10) 4-{[7-(5-methyl-2-furyl)-2-naphthyl]oxy}pyridine;
11) N-(5-bromo-1,3,4-oxadiazol-2-yl)-4-hydroxy-2-oxo-6-phenyl-2H-pyran-3-carboxamide;
12) 4-hydroxy-N-(5-methyl-2-furyl)-2-oxo-6-phenyl-2H-pyran-3-carboxamide;
13) 3-[(E)-2-(5-bromo-1,3,4-thiadiazol-2-yl)ethenyl]-4-hydroxy-6-phenyl-2H-pyran-2-one;
14) N-(5-bromo-1,3,thiadiazol-2-yl)-4-hydroxy-2-oxo-6-phenyl-2H-pyran-3-carboxamide;
15) 5-[(3-amino-1H-1,2,4-triazol-5-yl)methyl]-3-[3-fluoro-4-(4-morpholinyl)phenyl]-1,3-oxazolidin-2-one;
16) 4-[(3-amino-1H-1,2,4-triazol-5-yl)methyl]-1-[3-fluoro-4-(4-morpholinyl)phenyl]-2-imidazolidinone;
17) 1-benzhydryl-4-(5-bromo-2-furoyl)piperazine;
18) 1-benzhydryl-4-[(5-methyl-2-thienyl)carbonyl]piperazine;
43) benzyl (2E)-2-[1-(4-methyl-2-thienyl)ethylidene]hydrazinecarboxylate;
44) 2-(4-chlorophenyl)-6-methyl-5-(5-methyl-1,3,4-oxadiazol-2-yl)[-1,3]thiazolo[3,2-b][1,2,4]triazole;
45) N-(5-methyl-3-isoxazolyl)-N′-[(5-phenyl-1,3,4-oxadiazol-2-yl)carbonyl]urea;
46) N-[3-(2-{[(5-chloro-2-thienyl)methyl]sulfonyl}hydrazino)-3-oxopropyl]benzenesulfonamide5-[3-(4-phenoxyphenyl)propyl]-1,3,4-oxadiazol-2-ol;
47) N-(3-methyl-5-isoxazolyl)-4-phenoxybenzamide;
48) 4-hydroxy-N-(3-methyl-5-isoxazolyl)-2-oxo-6-phenoxy-2H-pyran-3-carboxamide;
49) 2-phenoxy-N′-[(Z)-phenyl(2-thienyl)methylidene]benzohydrazide;
50) 2-anilino-N′-[(Z)-2-furyl(phenyl)methylidene]benzohydrazide;
51) 4-[(Z)-1-(3-methyl-5-isoxazolyl)-2-phenylethenyl]phenyl 2-(1-pyrrolidinyl)ethyl ether;
52) 5-methyl-2-furaldehyde [(3Z)-2-oxo-1-(4-pyridinyl)-1,2-dihydro-3H-indol-3-ylidene]hydrazone;
53) (2Z)-N-[(5-methyl-2-furyl)methyl]-2-[2-oxo-1-(4-pyridinyl)-1,2-dihydro-3H-indol-3-ylidene]ethanamide;
54) (2Z)-N-[(3-methyl-5-isoxazolyl)methyl]-2-[2-oxo-1-(4-pyridinyl)-1,2-dihydro-3H-indol-3-ylidene]ethanamide;
32) (2-chloro-1,3-thiazol-5-yl)methyl 4-(4-morpholinylsulfonyl)phenyl ether;
33) N-(4,5-dihydronaphtho[1,2-d][1,3]thiazol-2-yl)-N-(4-phenoxybutyl)methanesulfonamide;
34) N-(6-methoxy-4,5-dihydronaphtho[1,2-d][1,3]thiazol-2-yl)-N-[2-(1-methyl-3-phenylpropoxy)ethyl]acetamide;
35) 4-{2-[(5-methyl-2-furyl)methoxy]benzylidene}-1-(4-pyridinylsulfonyl)piperidine;
36) 4-{2-[(5-bromo-2-furyl)methoxy]benzylidene}-1-isonicotinoylpiperidine;
37) N-(4,5-dihydronaphtho[1,2-d][1,3]thiazol-2-yl)-N-(4-phenylpentyl)acetamide;
38) N-(4,5-dihydro-3H-naphtho[1,2-d]imidazol-2-yl)-N-[2-(2-phenylethoxy)ethyl]methanesulfonamide;
39) N′-[(Z)-(5-methyl-2-furyl)(2-pyridinyl)methylidene]-2-phenoxybenzohydrazide;
or a pharmaceutically acceptable salt thereof.

21. A pharmaceutical composition comprising a compound of formula (I) as defined in claim 16, or a pharmaceutical acceptable salt thereof, and a carrier and/or diluent.

22. A method for inhibiting β-catenin/TCF-4 interaction in a patient in need thereof, the method comprising administering to said patient a therapeutically effective amount * of a compound of formula (I), or a pharmaceutically acceptable salt thereof, having the following formula: 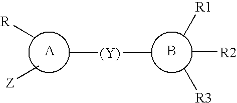
wherein:
(A) is a saturated, partially saturated, carbocyclic or heteroaromatic pentatomic ring;
(B) is a saturated, partially saturated, carbocyclic, aromatic or internally condensed ring;
(Y), in its shortest way, is a spacer consisting of about 4 to 9 chain atoms chosen independently from C, O, N and S, which may have independently different hybridization states, and wherein two to five adjacent atoms of the chain my be part of an optionally substituted aryl, heteroaryl or partially saturated aryl or heteroaryl ring system, which may be either isolated or include ring (B).
Z is a substituent selected independently from hydrogen, halogen, hydroxy, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl;
R is independently selected from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl, and a NHCORc or NHSO2Rc group wherein Rc is C1-C4 alkyl; or Z and R, taken together, form an optionally substituted, partially saturated monocyclic or bicyclic ring system; or Z and R, taken together, form an optionally substituted, partially saturated monocyclic or bicyclic ring system;
each of R1, R2 and R3, which may be independently the same or different, is chosen from hydrogen, halogen, cyano, a straight or branched C1-C4 alkyl group optionally substituted by 1 to 3 halogen atoms, a straight or branched C1-C4 alkoxy group, a N(RaRb) group wherein each of Ra and Rb independently is selected from hydrogen and C1-C4 alkyl; a NHCORc or NHSO2Rc group wherein Rc is C1-C4 alkyl; and a C5-C6 cycloalkyloxy or aryloxy group.

23. A method according to claim 22, for preventing and treating proliferative disorders, including cancer, in inhibiting cancer metastasis, in treating Alzheimer's disease and in modulating hair growth.

24. A method according to claim 22, for preventing and treating colorectal carcinoma, melanoma, liver carcinoma, breast cancer and prostatic cancer.
